(19) United States
(12) Patent Application Publication
(10) Pub. No.: US 2008/0160520 A1
(43) Pub. Date: Jul. 3, 2008

(54) Methods and Composition for Diagnosing and Treating Cancer

(76) Inventors: Angelika Amon, Cambridge, MA (US); Eduardo Torres, Cambridge, MA (US); Brett Williams, Cambridge, MA (US); Maitreya Dunham, Princeton, NJ (US)

Correspondence Address: MINTZ, LEVIN, COHN, FERRIS, GLOVSKY AND POPEO, P.C;ATTN: PATENT INTAKE CUSTOMER NO. 30623, ONE FINANCIAL CENTER, BOSTON, MA, 02111, US

(21) Appl. No.: 11/767,200

(22) Filed: Jun. 22, 2007

Related U.S. Application Data
(60) Provisional application No. 60/815,692, filed on Jun. 22, 2006.

(51) Int. Cl. C12Q 1/68; C12Q 1/37; C12N 5/06; C12Q 1/02

(52) U.S. Cl. 435/6; 435/24; 435/375; 435/29

(57) ABSTRACT

The present invention provides methods of detecting and treating cancer.

### RELATED APPLICATIONS

This application claims the benefit of U.S. Ser. No. 60/815,692, filed Jun. 22, 2006, which is incorporated herein by reference in its entirety.

### GOVERNMENT INTEREST

This invention was supported in part by National Institutes of Health grant GM56800. The United States government may have certain rights in the invention.

### FIELD OF THE INVENTION

The invention relates to generally to the detection and treatment of cancer.

### BACKGROUND OF THE INVENTION

Human growth and development requires the spatial and temporal regulation of cell differentiation, cell proliferation, and apoptosis. These processes coordinately control reproduction, aging, embryogenesis, morphogenesis, organogenesis, and tissue repair and maintenance. At the cellular level, growth and development is governed by the cell's decision to enter into or exit from the cell division cycle and by the cell's commitment to a terminally differentiated state. These decisions are made by the cell in response to extracellular signals and other environmental cues it receives.

Cell division is the fundamental process by which all living things grow and reproduce. In unicellular organisms such as yeast and bacteria, each cell division doubles the number of organisms. In multicellular species many rounds of cell division are required to replace cells lost by wear or by programmed cell death, and for cell differentiation to produce a new tissue or organ. Progression through the cell cycle is governed by the intricate interactions of protein complexes. This regulation depends upon the appropriate expression of proteins that control cell cycle progression in response to extracellular signals, such as growth factors and other mitogens, and intracellular cues, such as DNA damage or nutrient starvation. Molecules which directly or indirectly modulate cell cycle progression fall into several categories, including cyclins, cyclin-dependent protein kinases, growth factors and their receptors, second messenger and signal transduction proteins, oncogene products, and tumor-suppressor proteins.

Details of the cell division cycle may vary, but the basic process consists of three principle events. The first event, interphase, involves preparations for cell division, replication of the DNA, and production of essential proteins. In the second event, mitosis, the nuclear material is divided and separates to opposite sides of the cell. The final event, cytokinesis, is division and fission of the cell cytoplasm. The sequence and timing of cell cycle transitions is under the control of the cell cycle regulation system that controls the process by positive or negative regulatory circuits at various checkpoints.

Mitosis marks the end of interphase and concludes with the onset of cytokinesis. There are four stages in mitosis, occurring in the following order: prophase, metaphase, anaphase and telophase. Prophase includes the formation of bi-polar mitotic spindles, composed of microtubules that originate from polar mitotic centers. Furthermore, structural rearrangements occur ensuring appropriate distribution of cellular components between daughter cells. Breakdown of interphase structures into smaller subunits is common. The nuclear envelope breaks into vesicles, and nuclear lamins are disassembled. Subsequent phosphorylation of these lamins occurs and is maintained until telophase, at which time the nuclear lamina structure is reformed. During prophase, the nuclear material condenses and develops kinetochore fibers that aid in its physical attachment to the mitotic spindles. The ensuing movement of the nuclear material to opposite poles along the mitotic spindles occurs during anaphase. Telophase includes the disappearance of the mitotic spindles and kinetochore fibers from the nuclear material. Mitosis depends on the interaction of numerous proteins.

All key cell cycle transitions, including the entry and exit of a cell from mitosis, are dependent upon the activation and inhibition of cyclin-dependent kinases (Cdks). The Cdks are composed of a kinase subunit, Cdk, and an activating subunit, cyclin, in a complex that is subject to many levels of regulation. There appears to be a single Cdk in Saccharomyces cerevisiae and Schizosaccharomyces pombe whereas mammals have a variety of specialized Cdks. Cyclins act by binding to and activating cyclin-dependent protein kinases which then phosphorylate and activate selected proteins involved in the mitotic process. The Cdk-cyclin complex is both positively and negatively regulated by phosphorylation, and by targeted degradation involving molecules such as CDC4 and CDC53. In addition, Cdks are further regulated by binding to inhibitors and other proteins such as Suc1 that modify their specificity or accessibility to regulators (Patra, D. and W. G. Dunphy (1996) Genes Dev. 10: 1503-1515; and Mathias, N. et al. (1996) Mol. Cell Biol. 16:6634-6643).

Cyclins are degraded through the ubiquitin conjugation system (UCS), a major pathway for the degradation of cellular proteins in eukaryotic cells. The UCS mediates the elimination of abnormal proteins and regulates the half-lives of important regulatory proteins that control cellular processes such as gene transcription and cell cycle progression. The UCS is implicated in the degradation of mostif not all cyclins, oncoproteins, tumor suppressor genes such as p53, viral proteins, cell surface receptors associated with signal transduction, transcriptional regulators, and mutated or damaged proteins (Ciechanover, supra).

The process of ubiquitin conjugation and protein degradation occurs in five principle steps (Jentsch, S. (1992) Annu. Rev. Genet. 26:179-207). First ubiquitin (Ub), a small, heat stable protein is activated by a ubiquitin-activating enzyme (E1) in an ATP dependent reaction which binds the C-terminus of Ub to the thiol group of an internal cysteine residue in E1. Second, activated Ub is transferred to one of several Ub-conjugating enzymes (E2). Different ubiquitin-dependent proteolytic pathways employ structurally similar, but distinct ubiquitin-conjugating enzymes that are associated with recognition subunits that direct them to proteins carrying a particular degradation signal. Third, E2 transfers the Ub molecule through its C-terminal glycine to a member of the ubiquitin-protein ligase family, E3. Fourth, E3 transfers the Ub molecule to the target protein. Additional Ub molecules may be added to the target protein forming a multi-Ub chain structure. Fifth, the ubiquinated protein is then recognized and degraded by the proteasome, a large, multisubunit proteolytic enzyme complex, and Ub is released for re-utilization.

Prior to activation, Ub is usually expressed as a fusion protein composed of an N-terminal ubiquitin and a C-terminal extension protein (CEP) or as a polyubiquitin protein with Ub monomers attached head to tail. CEPs have characteristics of a variety of regulatory proteins; most are highly basic, contain up to 30% lysine and arginine residues, and have nucleic acid-binding domains (Monia, B. P. et al. (1989) J. Biol. Chem. 264:4093-4103). The fusion protein is an important intermediate that appears to mediate co-regulation of the cell's translational and protein degradation activities, as well as localization of the inactive enzyme to specific cellular sites. Once delivered, C-terminal hydrolases cleave the fusion protein to release a functional Ub (Mania et al., supra).

Abnormal activities of the UCS are implicated in a number of diseases and disorders. These include, e.g., cachexia (Llovera, M. et al. (1995) Int. J. Cancer 61:138-141), degradation of the tumor-suppressor protein, p53 (Ciechanover, supra), and neurodegeneration such as observed in Alzheimer's disease (Gregori, L. et al. (1994) Biochem. Biophys. Res. Commun. 203:1731-1738). Since ubiquitin conjugation is a rate-limiting step in antigen presentation, the ubiquitin degradation pathway may also have a critical role in the immune response (Grant, E. P. et al. (1995) J. Immunol. 155:3750-3758).

Cell cycle regulation not only involves proteins that replicate DNA and segregate it to the daughter cells but also involves numerous proteins whose function it is to ensure the precise order of cell cycle events. These control mechanisms are called checkpoints. For example, DNA damage (G₂) and DNA replication (S-phase) checkpoints arrest eukaryotic cells at the G₂/M transition. This arrest provides time for DNA repair or DNA replication to occur before entry into mitosis. Thus, the G₂/M checkpoint ensures that mitosis only occurs upon completion of DNA replication and in the absence of chromosomal damage. The Hus1 gene of Schizosaccharomyces pombe is a cell cycle checkpoint gene, as are the rad family of genes (e.g., rad1 and rad9) (Volkmer, E. and L. M. Kamitz (1999) J. Biol. Chem. 274:567-570; Kostrub C. F. et al. (1998) EMBO J. 17:2055-2066). These genes are involved in the mitotic checkpoint, and are induced by either DNA damage or blockage of replication. Induction of DNA damage or replication block leads to loss of function of the Hus1 gene and subsequent cell death. Human homologs have been identified for most of the rad genes, including ATM and AIR, the human homologs of rad3p. Mutations in the ATM gene are correlated with the severe congenital disease ataxia-telagiectasia (Savitsky, K. et al. (1995) Science 268: 1749-1753). The human Hus1 protein has been shown to act in a complex with rad1 protein which interacts with rad9, making them central components of a DNA damage-responsive protein complex of human cells (Volkner and Kamitz, supra). Examples of additional cell cycle regulatory proteins that are regulated by the DNA damage checkpoint include the histone deacetylases (HDACs). HDACs are involved in cell cycle regulation, and modulate chromatin structure. Human HDAC1 has been found to interact in vitro with the human Hus1 gene product, whose Schizosaccharomyces pombe homolog has been implicated in G₂/M checkpoint control (Cai, R. L. et al. (2000) J. Biol. Chem 275:27909-27916).

Cell cycle regulatory proteins play an important role in cell proliferation and cancer. For example, failures in the proper execution and timing of cell cycle events can lead to chromosome segregation defects resulting in aneuploidy or polyploidy. This genomic instability is characteristic of transformed cells (Luca, F. C. and M. Winey (1998) Mol. Biol. Cell. 9:2946) and a hallmark of solid tumors as virtually no solid tumor exists that does not show some alterations of the genome. With the vast majority of tumors this instability is expressed at the level of the chromosomal complement, and thus is detectable by cytogenetic approaches (Mitelman, F., Catalog of Chromosome Aberrations in Cancer, 5th Edition (New York: Wiley-Liss) (1994)). Further, cancers are characterized by continuous or uncontrolled cell proliferation. Strategies for treatment may involve either reestablishing control over cell cycle progression, or selectively stimulating apoptosis in cancerous cells (Nigg, E. A. (1995) BioEssays 17:471-480).

While a substantial amount of work has been performed in further understanding the cell cycle and how cancer is affected, no work has been done to understand why the problems occur. For example, cancer cells are known to be aneuploid. However, those of skill in the art have determined that aneuploidy or chromosomal rearrangement per se is not indicative of malignancy and many benign tumors can have an aberrant karyotype (Mitelman, 1994). It would be useful to determine the role aneuploidy plays in cancer progression and torn determine how this role can be monopolized to affect new cancer treatments.

### SUMMARY OF THE INVENTION

The invention provides biological markers to monitor the diagnosis and prognosis of cancer.

Cancer or a predisposition thereof is diagnosed in a subject by detecting the presence or an absence of a mutation in tumorgenesis-associated gene or polypeptide. The presence of a mutation indicates the presence of a cancer or a predisposition thereto in the subject. Whereas, the absence of a mutation indicates absence of a cancer or a predisposition thereto in the subject. Optionally, the level of the mutation in tumorgenesis-associated gene in the subject is compared to a control (i.e. standard) value. A higher level of mutation in tumorgenesis-associated gene or polypeptide in the test sample compared to the control sample indicates cancer in the subject. By higher level is meant at least a 2, 4, 5, 10-fold or higher value in the test sample compared to the control sample.

A tumorgenesis-associated gene or polypeptide include for Ubiquitin-specific protease, DNA helicase, a Subunit of the Set3C deacetylase complex and ORF YJL213W or a human homologue thereof. An Ubiquitin-specific protease includes for example UBP6 or a human homologue thereof such as USP14. A DNA helicase includes for example RAD3 or a human homologue thereof such as ERCC2. A Subunit of the Set3C deacetylase complex includes for example is SNT1 or a human homologue thereof such as FRS2 or SNTA1.

The mutation in the gene encoding an Ubiquitin-specific protease polypeptide is a stop codon which results in a less then full length polypeptide being expressed. For example, the mutation results in a C-terminal truncation of the polypeptide causing a decrease of peptidase activity of the polypeptide. The mutation in the gene encoding a DNA helicase polypeptide or a subunit of the Set3C deacetylase complex polypeptide is a point mutation. The point mutation results in an alteration, e.g., increase or decrease of polypeptide activity. The point mutation in DNA helicase polypeptide results in an amino acid change at position 148 resulting in an aspartic acid being changed to an asparagine. The point mutation in the Subunit of the Set3C deacetylase polypeptide results in an amino acid change at position 431 resulting in a leucine being changed to an arginine.

The mutation of ORF YJL213W is a mutation in the promoter region resulting in increased expression of the polypeptide encode by ORF YJL213W or the human homologue.

The sample is a biological sample obtained from the subject. The sample is for example, serum, blood plasma, ascites fluid, urine, a vaginal secretion or a tissue biopsy.

The tumorgenesis-associated polypeptide or gene is detected by any means known in the art. For example the tumorgenesis-associated polypeptide is detected electrophoretically or immunochemically. Immunochemical detection includes for example, radio-immunoassay, immunofluorescence assay, or enzyme-linked immunosorbant assay. The tumorgenesis-associated nucleic acid or gene is detected by the polymerase chain reaction, single nucleotide polymorphism (SNP) arrays, or interphase fluorescent in situ hybridization (FISH) analysis.

The subject has not been previously diagnoses as having cancer. Alternatively, the subject has been diagnosed with cancer. Optionally, the subject has been previously treated for cancer.

Also included in the invention is a method of decreasing cancer cell growth by contacting the tumor cell with a glucose transporter inhibitor, an RNA polymerases inhibitor, a protein synthesis inhibitor or a HSP90 inhibitor (e.g. geldanamycin). Protein synthesis inhibitors include for example, cycloheximide, hygromycin or rapamycin.

Alternatively, cancer cell growth decreased contacting the cancer cell with a compound that increases the expression or activity of a non-mutated UBP6, RAD3 or SNT1 polypeptide or human homologue thereof, decrease intracellular protein degradation; or decreases the expression of activity of the polypeptide encoded by ORF YJL213W or a human homologue thereof.

The cell is an aneuploid cell. The cell is contacted in vivo, in vitro or ex vivo. The methods treat or alleviate a symptom of cancer in a subject.

In a further aspect the invention provides a method for screening for an inhibitor of tumor cell proliferation or viability by contacting an aneuploid cell with a candidate compound. Cell proliferation or viability is measured. A decrease in aneuploid cell proliferation or viability in the presence of the compound, as compared to the absence of the compound, indicates that the compound is an inhibitor of tumor cell proliferation or viability. Alternatively, a diploid cell is also contacted with the candidate compared. A decrease in aneuploid cell proliferation or viability, as compared to the diploid cell indicates that the compound is a specific inhibitor of tumor cell proliferation or viability.

The aneuploid cell displays wild-type physiology. For example, the aneuploid cell has at least one mutation in a Ubiquitin-specific protease gene; a DNA helicase gene; a Subunit of the Set3C deacetylase complex gene or a promoter of ORF YJL213W or a human homologue thereof.

Also included in the invention are the compounds identified by the methods.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1A is a schematic illustration of the results of gene expression analysis demonstrating the effects of aneuploidy on gene expression Experiments (columns) are ordered by the number of the chromosome that is present in two copies. Data were renormalized to account for the disome. The columns labeled “wt” are biological replicates. The arrow points to the genomic location of HXT6 and HXT7.

FIG. 1B is a schematic illustration of hierarchically clustered gene expression data obtained from strains grown in batch cultures. Data from (A) were filtered for genes changing by greater than 1.8-fold on at least two arrays.

FIG. 1C is a schematic illustration of hierarchically clustered gene expression data obtained from strains grown in a chemostat under phosphate-limiting conditions.

FIG. 1D is a pie-chart representation of genes changing expression significantly in at least 10/14 disomic strains grown under phosphate-limiting conditions grouped by GO terms. Full GO results, including genes annotated to each term, can be found in Table S4.

FIG. 2 is a graphical representation showing the delay in G1 of the cell cycle in aneuploid cells. Wild type cells (A11311), cells disomic for chromosome IV (A12687), disomic for chromosome XIII (A12695) and disomic for chromosome VIII and XIV (A15615) all carrying a CLN2-HA fusion with the exception of strain A15615 were arrested in G1 with a-factor pheromone and released from the block as described in (10). Samples were taken at indicated times to determine the percentage of budded cells (A), DNA content (B), the percentage of cells with metaphase and anaphase spindles (C), and the amount of CLN2 RNA (D) and Cln2 protein (E). ACT1 was used as a loading control in Northern blots (D). Pgk1 was used as loading control in Western blots (E). In strain A15615, we only examined CLN2 RNA levels because chromosome XIV is not marked in this strain and we were therefore not able to select for the presence of two copies of this chromosome when introducing the Cln2-HA allele.

FIGS. 3 A and B are graphs showing increased glucose uptake in aneuploid strains. Wild type and aneuploid strains were grown in −his +G418 medium and OD600 was measured at the indicated times.

FIG. 3C shows the edibility of wild-type cells and aneuploid strains described in (A) to form colonies after being grown in −his +G418 medium to saturation. Samples were taken at the indicated times, and cells were plated on −his +G418 plates and the number of colonies was determined. Note that only cells disomic for chromosome II (A12685) and XV (A12697) maintained a high viability during stationary phase.

FIGS. 3 D and E are graphs showing glucose utilization of wild-type cells and cells disomic for chromosome IV (A12687; D) or chromosome XIV (A13979; E) or XI+XVI (A12699; E) grown to log phase.

FIG. 4A show the effects of increased gene dosage on protein abundance. Arp5, Tcp1, Cdc28, Rpa1, Mre11 and Rps2 and Rp132 proteins were examined in wild type cells, in cells disomic for the chromosome the encoding gene is located on and a control disome by Western blot analysis. The disome the encoding gene of interest is located on is highlighted in red. RNA levels of the gene product of interest are shown as a log₂ ratio of wild type of an average of two microarray analyses below the blot. 50 (8×), 25 (4×), 13 (2×) and 6 μg (1×) of extract were loaded. Nop1 was used as a loading control. Arp5 protein and RNA levels were analyzed in WT (A11311), Dis II (A12685) and Dis XIV (A13979); Tcp1 in WT (A11311), Dis II (A12685) and Dis IV (A12687); Cdc28 in WT (A11311), Dis II (A12685) and IV (A12687); Rpa1 in WT (A11311), Dis I (A12683) and Dis V (14479), Mre1 1 in WT (A11311), Dis I (A12683) and Dis XIII (A12695) and Rps2 and Rp132 in WT (A11311), Dis II (A12685) and Dis XV (A12697).

FIG. 4B shows the proliferative capability of disomes in the presence of Thiolutin.

FIG. 4C shows the proliferative capability of disomes in the presence of Cycloheximide.

FIG. 4D shows the proliferative capability of disomes in the presence of Hygromycin and Rapamycin.

FIG. 4E shows the proliferative capability of disomes at 37° C.

FIG. 4B shows the proliferative capability of disomes in the presence of MG132 and Geldanamycin (F).

FIG. 5A is a graphical illustration showing gene expression of YAC-containing strains grown under phosphate-limiting conditions. The gene expression pattern is shown for the 397 genes identified as changed in aneuploid strains grown under phosphate-limiting conditions. Data for wild-type cells and cells disomic for chromosome IX are from FIG. 1C and are shown for comparison. Data are provided in Table S5. The order of strains (from the left): A 11311, A13975, A16854 (this strain contains a truncated version of YAC-1), A17392, A17393, A17394, A17397, A16851.

FIG. 5A shows the behavior of YAC-carrying strains and aneuploid strains in the presence of high temperature (37° C., 39° C.), Thiolutin, Cycloheximide, Rapamycin and Hygromycin. Strains (from the top): A11311, A16850, A17392, A17393, A17394, A13628, A17396, A17397, A16851.

FIGS. 5 C and D shows wild type cells (A11311), and cells carrying the yeast artificial chromosome YAC-1 (A16850), YAC-7 (A16851), YAC-2 (A17392), YAC-3 (A17393), YAC-4 (A17394), YAC-5 (A17396) or YAC-6 (A17397) released from a pheromone-induced G1 arrest as described in FIG. 2. At the indicated times samples were taken to determine the percentage of budded cells (C) and DNA content (D).

FIG. 6 is a schematic representation showing the strategy to generate aneuploid yeast strains.

FIG. 7 A is a schematic representation showing CGH analysis of aneuploid strains. Each box represents the genome of an aneuploid yeast strain. Data points are ordered according to their chromosomal coordinates, starting from the left with the gene most distally located on the left arm of chromosome I. DNA content is shown as the running 5 gene median copy number of aneuploid strains, as measured by comparison with wild type. The chromosomes present at two copies are shown in red. The log₂ ratio data are provided in Table S5.

FIG. 7 B are bar graphs showing the raw data for the log₂ ratio of the HXT6 gene copy number (top) and RNA levels (bottom) in disomic strains compared to wild type strain.

FIG. 8 A is a schematic representation showing clustered gene expression pattern of aneuploid strains with all genes weighted equally. Gene expression data shown in FIG. 1B were hierarchically clustered with equal weights given to all genes.

FIG. 8 B is a schematic representation showing clustered gene expression pattern of aneuploid strains with all genes weighted equally. Gene expression data from FIG. 1C were hierarchically clustered with equal weights given to all genes.

FIG. 9 A are bar charts showing cell volume (top) and doubling times (bottom) of a wild type haploid and haploid cells carrying an extra chromosome grown in YEPD at 22° C. For the doubling time analysis, culture densities were measured at OD₆₀₀ every 2 hours from OD₆₀₀=0.2 to OD₆₀₀=1. OD₆₀₀ measurements were plotted as a function of time and fitted to an exponential growth curve. Data are shown as mean +/−SD (n=3).

FIG. 9 B are bar charts showing cell volume (top) and doubling times (bottom) of a wild type haploid and haploid cells carrying an extra chromosome grown in −his G418 medium at 22° C. Note that the degree by which the doubling time increases in aneuploids compared to wild-type is greater in −his +G418 medium than in YEPD, possibly due to the presence of G418 in the medium and/or the lack of histidine.

FIG. 9 C are bar charts showing cell volume (top) and doubling times (bottom) of a wild type diploid and diploid cells carrying an extra chromosome grown in YEPD medium at 22° C.

FIG. 9 D are bar charts showing cell volume (top) and doubling times (bottom) of a wild type diploid and diploid cells carrying an extra chromosome grown in −his −ura G418 medium at 22° C. Note that the degree by which the doubling time increases in aneuploids compared to wild-type is greater in −his −ura +G418 medium than in YEPD, possibly due to the presence of G418 in the medium and/or the lack of histidine and uracil. Also note that the doubling times of disomes and trisomes can only be compared between (A) and (C) as the drop out media used in (B) and (D) are note the same.

FIG. 9 E are bar charts showing plating efficiency of a wild type haploid and haploid cells carrying an extra chromosome grown in YEPD at 22° C.

FIG. 9 F are line graphs showing competition experiments between Leu+ wild type cells (A17413, closed black squares) and Trp1+ wild type cells (A17414, open squares). Another competition experiment determined the fitness of a strain disomic for chromosome I and Trp+ (A17416, opened triangles) when co-cultured with a Leu+ wild type strain (A17413, closed triangles).

FIG. 9 G are line graphs showing competition experiments between a Trp1+ wild type strain (A17414, closed squares) and Ura+ wild type strain (A17415, open squares). Another competition experiment determined the fitness of a strain disomic for chromosome II and Ura+ (A17417, opened triangles) when co-cultured with a Trp1+ wild type strain (A17413, closed triangles).

FIG. 10 are graphs showing ell cycle analysis of aneuploid cells. Wild type (A11311) and aneuploid strains were arrested in G1 in YEPD with 5 μg/ml α-factor and released from the block after 3 hours at room temp. Samples were taken at indicated times to determine the percentage of budded cells (top panel), DNA content (middle panel) and the percentage of cells with metaphase and anaphase spindles (bottom panel). The following strains were analyzed: Dis I (A12683), Dis II (A12685), Dis V (A14479), Dis VIII (A13628), Dis IX (A13975), Dis X (A12689), Dis XI (A13771), Dis XII (A12693), Dis XIV (A13979), Dis XV (A12697), Dis XVI (A12700), Dis XI+XVI (A12699), Dis XI+XV (A12691), Dis I+VI+XIII (A15619), Dis VIII+XV (A15579), Dis V+IX (A16308) and Dis V+VII (A16309).

FIG. 11 is a graph showing linear regression analysis demonstrating a correlation between the G1 delay and the amount of extra yeast DNA present in aneuploids. A linear correlation fits all the delays with an R²=0.41 (not shown). When aneuploid strains containing chromosomes II and XV (data points in red) are omitted the data fits a linear correlation with an R²=0.66 (black line). Data points for the strains carrying YACs with mouse or human DNA are shown in green.

FIGS. 12A-J demonstrate the effects of increased gene dosage on protein abundance Nop1, Pup3, Pre6, Hht1, Rpt1, Lcb4, Elp3, Eaf3, Yaf9 and Fcy1 proteins (top) were examined in wild type cells, in cells disomic for the chromosome the encoding gene is located on and a control disome by Western blot analysis. The disome the encoding gene of interest is located on is highlighted in red. RNA levels of the gene product of interest are shown as a log₂ ratio of wild type of an average of two microarray analyses below the blot. 50 (8×), 25 (4×), 13 (2×) and 6 μg (1×) of extract were loaded. Pgk1 and Nop1 were used as a loading control.

(A) Nop1 protein and RNA levels are shown in WT (A11311), Dis IV (A12687) and Dis XIII (A12695).

(B) Pup3 protein and RNA levels are shown in WT (A11311), Dis V (A14479) and Dis XI (A13771).

(C) Pre6 protein and RNA levels are shown in WT (A11311), Dis IV (A12687) and Dis XV (A12697).

(D) Hht1 protein and RNA levels are shown in WT (A11311), Dis I (A12683) and II (A12685).

(E) Rpt1 protein and RNA levels are shown in WT (A11311), Dis V (A14479) and XI (A13771).

(F) Lcb4 protein and RNA levels are shown in WT (A11311), Dis XV (A12697) and XVI (A12700).

(G) Elp3 protein and RNA levels are shown in WT (A11311), Dis XV (A12697) and XVI (A12700).

(H) Eaf3 protein and RNA levels are shown in WT (A11311), Dis XV (A12697) and XVI (A12700).

(I) Yaf9 protein and RNA levels are shown in WT (A11311), Dis IV (A12687) and XIV (A13979).

(J) Fcy1 protein and RNA levels are shown in WT (A11311), Dis VIII (A13628) and XVI (A12700).

FIG. 13 A-I show the effects of carbon source, cell cycle inhibitors and authophagy inhibitors on aneuploid strains.

(A) Wild type haploid (A11311) and diploid (A702) strains plated on YPD at 30° C. and 37° C. Also plated on YPD at 30° C. containing 20 μg/ml of Thiolutin, 10 nM Rapamycin and 0.05 μg/ml Cycloheximide.

(B-I) Wild type strains and aneuploid strains were plated on medium containing increasing concentrations of Hydroxyurea (B), 3% glycerol (YPG) or 2% raffinose and galactose (Raf/Gal) as the sole carbon source (C), medium containing 1 mM chloroquine (D), 0.5 mg/ml azetidine 2-carboxylic acid (E), 10 and 20 μg/ml benomyl (F), 1 mM hydrogen peroxide (G), increasing concentrations of Thiolutin (H), 60 μg/ml of 6-Azauracil, YEP medium containing glycerol and ethanol as the sole carbon source (YPGE) and increasing concentrations of Oligomycin (I).

### DETAILED DESCRIPTION OF THE INVENTION

The invention is based in part upon the discovery that aneuploid yeast strains share a number of phenotypes that are distinct from diploid cells and are independent of the identity of the individual extra chromosomes. Specifically, aneuploid cells have defects in cell cycle progression, increased glucose uptake and increased sensitivity to conditions interfering with protein synthesis and protein folding. More specifically, the invention is based upon the identification of specific mutations in aneuploid cells which allows the cells to tolerate aneuploidy.

Aneuploidy is a condition frequently found in cancer cells and share several properties with yeast cells carrying additional chromosomes. For example, proliferation of both types of cells is impaired in the presence of protein synthesis inhibitors (27) and geldanamycin (28) and both exhibit increased glucose uptake (29). Until now it has been thought that the proliferative advantage of tumor cells which makes them independent of growth control was the direct result of the aneuploidy which leads to the cell carrying extra copies of oncogenes. In contrast, the data described herein demonstrate that aneuploidy results in a proliferative disadvantage and that this disadvantage needs to be overcome during cancer formation. This proliferative disadvantage is overcome in part by acquiring specific mutations in the genes encoding ubiquitin-specific protease, DNA helicase, a subunit of the Set3C deacetylase complex and in the promoter region of yeast ORF YJL213W. These mutations are summarized in Table A and are collectively referred to herein as “tumorgenesis-associated mutations” and the corresponding genes and polypeptides in which these mutation are found are referred to herein as “tumorgenesis-associated genes”, tumorgenesis-associated nucleic acids” “tumorgenesis-associated polynucleotides”, “tumorgenesis-associated polypeptides” or “tumorgenesis-associated proteins.” The genes have been previously described and are presented along with a database accession numbers.

TABLE A

| Yeast | | Nucleic Acid | Polypeptide | Human |
|---|---|---|---|---|
| Gene | Gene family | Mutation* | Mutation | Homologues |
| UBP6 | ubiquitin- | G→T at position | Stop codon | USP14 |
| | specific | 165825 of yeast | | |
| | protease | chromosome VI | | |
| RAD3 | DNA | G→A at position | D→N at | ERCC2 |
| | helicase | 527517 of yeast | position 148 | |
| | | chromosome V | | |
| SNT1 | subunit of | T→G at position | L→R at | FRS2, |
| | the Set3C | 187773 of yeast | position 431 | SNTA1 |
| | deacetylase | chromosome III | | |
| | complex | | | |
| ORF | unknown | C→G at position | Upregulation | unknown |
| YJL213W | | 31906 of yeast | of protein | |
| | | chromosome X | expression | |

*Position is based upon the location in the yeast genome

Accordingly, the invention provides methods of detecting and evaluating cancer in a subject by the detection of a mutation in a tumorgenesis-associated gene or tumorgenesis-associated polypeptide. Also provided by the inventions are methods of decreasing tumor cell growth and treating or alleviating a symptom of cancer by contacting a cell or administering to a subject a compound that modulates the expression of a tumorgenesis-associated gene, a tumorgenesis-associated polypeptide, a glucose transporter inhibitor, an RNA polymerase inhibitor, a protein synthesis inhibitor, or a HSP90 inhibitor. The methods disclosed herein are employed with subjects suspected of having cancer, to monitor subjects who have been previously diagnosed as having cancer, and to screen subjects who have not been previously diagnosed as having cancer.

The cell division cycle is a highly controlled process that generates two daughter cells of identical genetic make-up. Surveillance mechanisms known as checkpoints ensure that this process occurs with high fidelity. However, despite these surveillance mechanisms, chromosome mis-segregation occurs once every 5×10⁵ cell divisions in yeast (1) and on the order of once every 10⁴-10⁵ divisions in mammalian cells (2), producing a condition known as aneuploidy. More than a century ago, aneuploidy was postulated to be a common characteristic of cancer cells (3). Since then, it has been proposed that aneuploidy contributes to tumorigenesis by providing a mechanism by which oncogenes are gained or tumor suppressor genes are lost (4). Studies examining the effects of aneuploidy on cell proliferation in S. pombe (5), Drosophila (6), and of trisomy on cell proliferation in humans (7) suggest that aneuploidy can also interfere with cell proliferation. To address how aneuploidy affects the proliferation and the physiology of normal cells, a set of yeast strains in which each strain bears an extra copy of one or more of almost all of the yeast chromosomes were generated. Their characterization represents a comprehensive analysis of the effects of aneuploidy on cellular physiology and revealed that in addition to chromosome-specific phenotypes, aneuploid strains share a number of traits, pointing towards the existence of a general cellular response to aneuploidy.

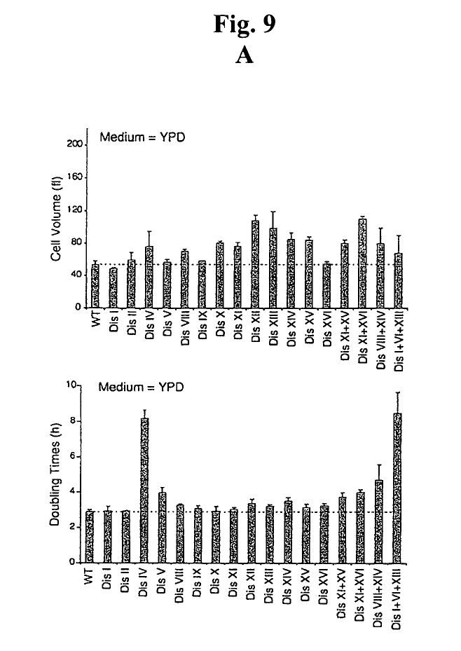

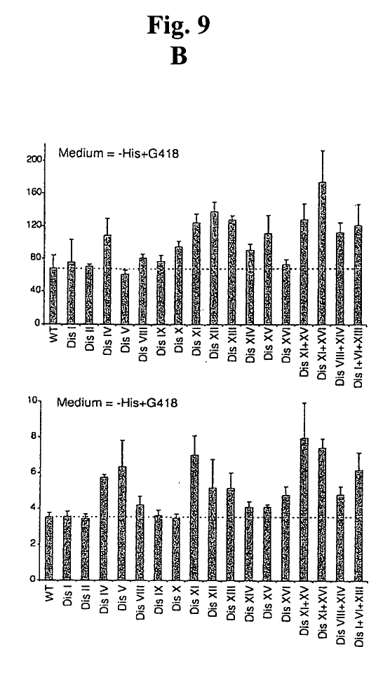

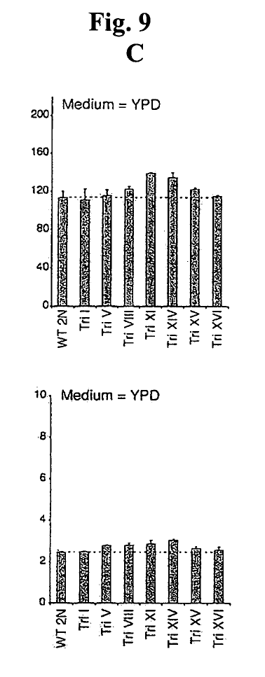

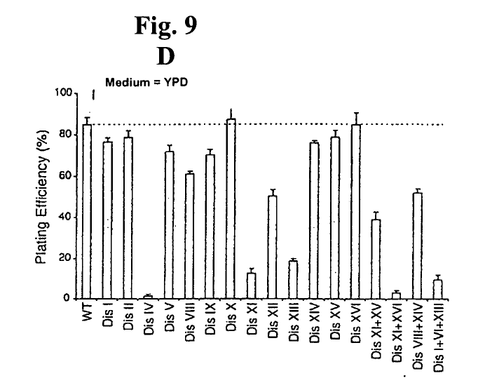

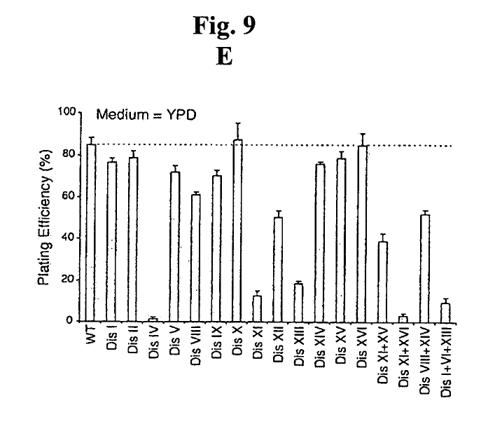

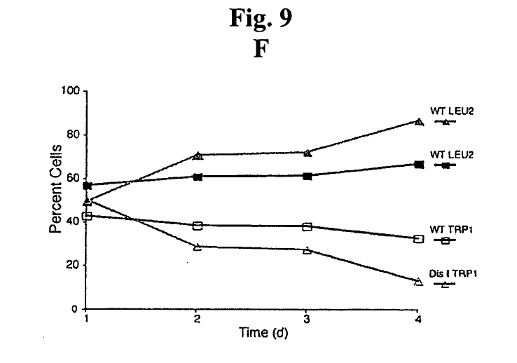

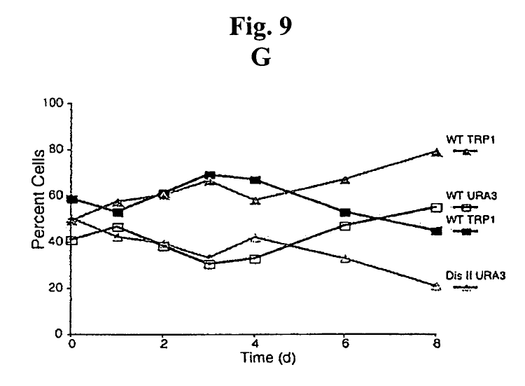

Specifically, the 20 aneuploid yeast strains that were analyzed shares several phenotypes. In contrast, diploid yeast cells did not exhibit these phenotypes (FIGS. 9 and 13A). This result not only shows that the duplication of the entire genome is not nearly as deleterious as the duplication of a subset of chromosomes but indicates that it is the genomic imbalance that results from aneuploidy that is responsible for the phenotypes we observe. The finding that the severity of the phenotypes shared by aneuploids is generally greater in strains disomic for large or multiple chromosomes supports this idea. This data further suggest that an increase in ploidy buffers the detrimental effects of the imbalances caused by aneuploidy. The phenotypes shared by aneuploids were generally less severe in trisomic than in disomic cells.

These studies indicated that most phenotypes common to aneuploids are caused by the additional yeast gene products. These findings together with the observation that disomy for the small chromosome VI is lethal (10) indicate that likely both the total amount of additional RNA and protein produced by aneuploids as well as specific gene products present on individual chromosomes contribute to the phenotypes shared by aneuploids.

Striking among the phenotypes shared by aneuploid yeast strains are those indicative of protein degradation and folding distress. These observations suggest that proteins synthesized from the additional chromosomes disrupt cellular physiology, interfering with metabolic pathways and other basic cellular processes. It is hypothesized the cells respond to this state of imbalance in a multi-layered fashion not dissimilar to that of a stress response. The cell's attempt to restore wild type physiology is reflected by the fact that although most genes present on the additional chromosomes are transcribed, the amounts of many proteins are not increased. Carbohydrate uptake is increased which could provide the energy needed to degrade protein and induce mechanisms that shield the cell from the effects of excess proteins or compensate for their effects. The delay in G1 might also be part of a response to this disruption in cellular homeostasis.

### Ubiquitin-Specific Protease

Ubiquitin-specific proteases (UBPs) are a family of unique hydrolases that specifically remove polypeptides covalently linked via peptide or isopeptide bonds to the C-terminal glycine of ubiquitin. UBPs help regulate the ubiquitin/26S proteolytic pathway by generating free ubiquitin monomers from their initial translational products, recycling ubiquitins during the breakdown of ubiquitin-protein conjugates, and/or by removing ubiquitin from specific targets and thus presumably preventing target degradation.

A point mutation in the yeast gene UBP6 was identified in aneuploid yeast cells that have overcome the proliferation disadvantage of aneuoploid cells. UBP6 is located on yeast chromosome VI from coordinates 165060 to 166559. The point mutation resulted in nucleotide change (G→T) at position 165623, resulting in a stop codon. This stop codon results in a truncated version of the ubiquitin-specific protease polypeptide. Specifically, the protein lacks the C-terminal peptidase activity, while retaining the N-terminal ubiquination-like domain.

### DNA Helicase

DNA helicase is an enzyme that aids in DNA synthesis by ‘unzipping’ the two strands of a DNA helix so that DNA polymerase can access the DNA to add nucleotides and effect copying.

Many cellular processes such as DNA replication, RNA transcription, DNA recombination, DNA repair, Ribosome biogenesis involve the separation of nucleic acid strands. Helicases are often utilized to separate strands of a DNA double helix or a self-annealed RNA molecule using the energy from ATP or GTP hydrolysis. They move incrementally along one nucleic acid strand of the duplex with directionality specific to each particular enzyme. There are many helicases (e.g., 24 in human cells) resulting from the great variety of processes in which strand separation must be catalyzed.

The common function of helicases accounts for the fact that they display a certain degree of amino acid sequence homology; they all possess common sequence motifs located in the interior of their primary sequence. These are thought to be specifically involved in ATP binding, ATP hydrolysis and translocation on the nucleic acid substrate. The variable portion of the amino acid sequence is related to the specific features of each helicase.

Based on the presence and the form of helicase motifs, helicases have been separated in 4 superfamilies and 2 smaller families.

A point mutation in the yeast gene RAD3 was identified in aneuploid yeast cells that have overcome the proliferation disadvantage of aneuoploid cells. Rad3 is located on yeast chromosome V from coordinates 527077 to 529413. The point mutation resulted in nucleotide change (G→A) at position 527517, resulting D→N amino acid change at position 148.

### Subunit of the SET3C Deacetylase Complex

SET3C is a NAD-dependent histone deacetylase. The Set3 complex (Set3C) includes two potential histone deacetylases, Hos2 and Hst1. Hos2 is a class I histone deacetylase like Rpd3 and the mammalian HDACs 1-3 (Rundlett et al. 1996). Hst1 is a member of the recently identified Sir2 class of NAD-dependent deacetylases (Imai et al. 2000; Landry et al. 2000; Smith et al. 2000) and has been linked previously to repression of sporulation genes (Xie et al. 1999; Lindgren et al. 2000). The other components of the complex are Snt1, YIL112w, Sif2 and Cpr1. IN budding the yeast Set3C has been implicated in establishing silenced chromatin and the repression of meiosis-specific genes. The human homologs of this deacetlyase are also involved in silencing transcription.

A point mutation in the yeast gene SNT1 was identified in aneuploid yeast cells that have overcome the proliferation disadvantage of aneuoploid cells. SNT1 is located on yeast yeast chromosome III from coordinates 186485 to 190165. The point mutation resulted in nucleotide change (T→G) at position 187773, resulting amino L→R acid change at position 148.

### ORF YJL213W

YJL213W is a protein of unknown function that may interact with ribosomes; periodically expressed during the yeast metabolic cycle; and is phosphorylated in vitro by the mitotic exit network (MEN) kinase complex, Dbf2p/Mob1p.

A point mutation in the yeast gene the upstream (e.g, in the promoter region) of YJL213W of was identified in aneuploid yeast cells that have overcome the proliferation disadvantage of aneuoploid cells. YJL213W is located on yeast yeast chromosome X from coordinates 32163 to 33158. The point mutation resulted in nucleotide change (C→G) at position 31906, resulting in the upregulation of the expression of the YJL213W protein.

### Diagnostic and Prognostic Methods

The invention provides diagnostic and prognostic methods for identifying a subject with cancer or a predisposition thereto.

Cancers or a predisposition of developing cancer are detected by examining the presence or absence of a mutation in tumorgenesis-associated gene or tumorgenesis-associated polypeptide in a test population of cells (i.e., a patient derived sample). The presence of one or more tumorgenesis-associated mutation indicates that the subject has is predisposed to developing cancer. Whereas, the absence of a tumorgenesis-associated mutation indicates that the subject does not have cancer or predisposed to developing cancer.

In some aspects mutation in tumorgenesis-associated gene or tumorgenesis-associated polypeptide is determined in the test sample to provide a test value and the test value is compared to a standard value. By standard value is meant the level tumorgenesis-associated gene or tumorgenesis-associated polypeptide typically found in a population not having cancer. The standard value can be a range or an index. Alternatively, the standard value can be a database of test values from previously tested samples.

The difference in the standard value compared to the test value is statistically significant. By statistically significant is meant that the alteration is greater than what might be expected to happen by chance alone. Statistical significance is determined by method known in the art. For example statistical significance is determined by p-value. The p-values is a measure of probability that a difference between groups during an experiment happened by chance. (P(z≧z_{observed})). For example, a p-value of 0.01 means that there is a 1 in 100 chance the result occurred by chance. The lower the p-value, the more likely it is that the difference between groups was caused by treatment. An alteration is statistically significant if the p-value is at least 0.05. Preferably, the p-value is 0.04, 0.03, 0.02, 0.01, 0.005, 0.001 or less.

Cancer includes solid tumors and hematologic tumors. Cancers include for example, lung cancer, head or neck cancer, bladder cancer, kidney cancer, pancreatic cancer, brain cancer, liver cancer, colon cancer, stomach cancer, breast cancer, ovarian cancer, prostate cancer, testicular cancer, skin cancer, or leukemia.

The patient derived sample can be any tissue or fluid. Tissue samples include for example, paraffin imbedded tissue, frozen tissue, surgical fine needle aspirations, cells of the skin, muscle, lung, head and neck, esophagus, kidney, pancreas, mouth, throat, pharynx, larynx, esophagus, facia, brain, prostate, breast, endometrium, small intestine, blood cells, liver, testes, ovaries, uterus, cervix, colon, stomach, spleen, lymph node, bone marrow or kidney. Fluid samples include of example, bronchial brushes, bronchial washes, bronchial ravages, peripheral blood lymphocytes, lymph fluid, ascites, serous fluid, pleural effusion, sputum, cerebrospinal fluid, lacrimal fluid, esophageal washes, and stool or urinary specimens such as bladder washing and urine.

Mutation in the genes disclosed herein is determined at the protein or nucleic acid level using methods known in the art. Mutation in nucleic acids are detected for example by Hybridization-based Assays and Amplification-based Assays. Mutations are is also determined at the protein level, i.e., by measuring the levels of polypeptides encoded by the gene products described herein. Such methods are well known in the art and include, e.g. immunoassays based on antibodies to proteins encoded by the genes.

The subject is preferably a mammal. The mammal is, e.g., a human, non-human primate, mouse, rat, dog, cat, horse, or cow. The subject has been previously diagnosed as having cancer, and possibly has already undergone treatment for the cancer. Alternatively, the subject has not been previously diagnosed as having cancer. The present invention is useful with all patients at risk for cancer.

Diagnosis of cancer is made through methods known in the art for a particular cancer such as patient history and physical examination along with diagnostic testing such as blood test, urinalysis, and tumor markers (e.g., PSA, PAP, CA125, CES, AFP and HCG)

### Therapeutic Methods

The invention provides a method for decreasing tumor cell growth, treating or alleviating a symptom of cancer in a subject by contacting the cell or administering to a subject a compound that increasing the expression or activity of a non-mutated ubiquitin-specific protease polypeptide, DNA helicase polypeptide, or a subunit of the Set3C deacetylase complex polypeptide, decreases intracellular protein degradation or decreases the expression or activity of the polypeptide encoded by ORFYJL213W or human homologue thereof. Alternatively, tumor cell growth is decreased and/or cancer is treated by contacting the cell or administering to a subject a glucose transporter inhibitor, an RNA polymerase inhibitor, a transcription inhibitor, a protein synthesis inhibitor or a HSP90 inhibitor.

Therapeutic compounds are administered prophylactically or therapeutically to subject suffering from, or at risk of or susceptible to developing, cancer. Such subjects are identified using standard clinical methods.

The therapeutic method includes increasing the expression, or function, or both of one or more gene products of a non-mutated ubiquitin-specific protease polypeptide, DNA helicase polypeptide, or a subunit of the Set3C deacetylase in a subject or cell relative to a normal subject or cells of the same tissue type. In these methods, the subject is treated with an effective amount of a compound, which increases the amount of one of more of the genes or polypeptides in the subject. Administration can be systemic or local. Therapeutic compounds include a polypeptide product (e.g., a ubiquitin-specific protease polypeptide, DNA helicase polypeptide, or a subunit of the Set3C deacetylase), or a biologically active fragment thereof, and a nucleic acid encoding polypeptide and having expression control elements permitting expression in the cell or subject. Administration of such compounds counters the effects of mutated genes in the subject and improves the clinical condition of the subject

The method also includes decreasing the expression, or function, or both, of the polypeptide encoded by ORF YJL213W or human homologue thereof whose expression is aberrantly increased (“overexpressed gene”) in cancer cells. Expression is inhibited in any of several ways known in the art. For example, expression is inhibited by administering to the subject a nucleic acid that inhibits, or antagonizes, the expression of the ORF YJL213W, e.g., an antisense oligonucleotide or siRNA which disrupts expression of the cancer-associated gene or genes.

Alternatively, function of one or more gene product of ORF YJL213W is inhibited by administering a compound that binds to or otherwise inhibits the function of the ORF YJL213W gene products. For example, the compound is an antibody which binds to the overexpressed gene product.

The method further includes decreasing intracellular protein degradation. Intracellular protein degradation is decreased by methods known in the art such as by decreasing ubiquination. For example, ubiquination is inhibited by administering to the subject a nucleic acid that inhibits, or antagonizes, the expression of ubiquitin or a polypeptide in the ubiquination pathways, e.g., an antisense oligonucleotide or siRNA which disrupts expression of the genes encoding these polypeptides.

Once the cancer cells have been modified to react as normal aneuploid cells the cells become susceptible to the normal sensitivities of aneuploid cells. Such as the aneuploid cells are sensitive to temperature, compounds that interfere with protein synthesis, geldanamycin (an inhibitor or HSP90, which is important to help fold misfolded proteins), hygromycin B, rapamycin, and cycloheximide. Accordingly in some aspects the cells or subjects are further contacted with a glucose transporter inhibitor, an RNA polymerase inhibitor, a transcription inhibitor, a protein synthesis inhibitor or a HSP90 inhibitor.

These modulatory methods are performed ex vivo or in vitro (e.g., by culturing the cell with the agent) or, alternatively, in vivo (e.g., by administering the agent to a subject). The method involves administering a protein or combination of proteins, a nucleic acid molecule or combination of nucleic acid molecules, or a combination of one or more nucleic acids and one or more proteins, as therapy to counteract aberrant expression or activity of the differentially expressed genes.

Diseases and disorders that are characterized by increased (relative to a subject not suffering from the disease or disorder) levels or biological activity of the genes may be treated with therapeutics that antagonize (i.e., reduce or inhibit) activity of the overexpressed gene or genes. Therapeutics that antagonize activity are administered therapeutically or prophylactically.

Therapeutics that may be utilized include, e.g., (i) a polypeptide, or analogs, derivatives, fragments or homologs thereof, of the overexpressed or underexpressed sequence or sequences; (ii) antibodies to the overexpressed or underexpressed sequence or sequences; (iii) nucleic acids encoding the over or underexpressed sequence or sequences; (iv) antisense nucleic acids or nucleic acids that are “dysfunctional” (i.e., due to a heterologous insertion within the coding sequences of coding sequences of one or more overexpressed or underexpressed sequences); or (v) modulators (i.e., inhibitors, agonists and antagonists that alter the interaction between an over/underexpressed polypeptide and its binding partner. The dysfunctional antisense molecule is utilized to “knockout” endogenous function of a polypeptide by homologous recombination (see, e.g., Capecchi, Science 244: 1288-1292 1989). The siRNA is designed by methods known in the art to bind to gene transcripts and prevent translation into proteins.

Diseases and disorders that are characterized by decreased (relative to a subject not suffering from the disease or disorder) levels or biological activity may be treated with therapeutics that increase (i.e., are agonists to) activity. Therapeutics that upregulate activity may be administered in a therapeutic or prophylactic manner. Therapeutics that may be utilized include, but are not limited to, a polypeptide (or analogs, derivatives, fragments or homologs thereof) or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of a gene whose expression is altered). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

Prophylactic administration occurs prior to the manifestation of overt clinical symptoms of disease, such that a disease or disorder is prevented or, alternatively, delayed in its progression.

Therapeutic methods include contacting a cell with an agent that modulates one or more of the activities of the gene products. An agent that modulates protein activity includes a nucleic acid or a protein, a naturally-occurring cognate ligand of these proteins, a peptide, a peptidomimetic, or other small molecule. For example, the agent stimulates one or more protein activities.

### Screening Assays for Identifying Therapeutic Agents

The aneuploid yeast cells disclosed herein can also be used to identify candidate inhibitors of tumor cell proliferation. The method is based on screening a candidate therapeutic agent to determine whether the compound has an effect on cell proliferation and or cell viability.

In the method, a cell is exposed to a test agent or a combination of test agents (sequentially or consequentially) and cell proliferation or cell viability is measured. Cell proliferation or cell viability in the test population is compared cell proliferation in a reference cell population that is not exposed to the test agent.

Cell proliferation is measured by methods known in the art, such as bromodeoxyuridine incorporation. Cell viability is measured for example by trypan blue exclusion.

The aneuploid cell displays normal wild type physiology. By normal wild type physiology is mean that the cell does not display the aneuploid phenotype such as defects in cell cycle progression, increased glucose uptake and increased sensitivity to conditions interfering with protein synthesis and protein folding. For example, the cell has one or more tumorgenesis-associated mutations.

The cell population in the reference population is a cell population that is identical to the test population. For example, the reference cell population is an aneuploid cell displays normal wild type physiology. Alternatively, the reference cell population is a normal cell, i.e., diploid. This allows the identification of compounds that preferentially decreases cell proliferation and or viability in tumor cells that have one or more tumorgenesis-associated mutations compared to normal cells. Such compositions are particularly useful in treating cancer as they will be specific for tumor cells and have no deleterious effects on normal cells.

An agent effective in decreasing cell proliferation and/or increasing cell viability is deemed to lead to a clinical benefit such compounds are further tested for the ability to decrease tumor cell proliferation and/or viability in animals or test subjects.

Kits for Use in Diagnostic and/or Prognostic Applications

For use in diagnostic, research, and therapeutic applications suggested above, kits are also provided by the invention. In the diagnostic and research applications such kits may include any or all of the following: assay reagents, buffers, nucleic acids for detecting the target sequences and other hybridization probes and/or primers. A therapeutic product may include sterile saline or another pharmaceutically acceptable emulsion and suspension base.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### Pharmaceutical Preparations

The phrases “pharmaceutical” and “pharmacologically acceptable” refer to molecular entities and compositions that do not produce an adverse, allergic or other untoward reaction when administered to an animal, such as, for example, a human, as appropriate. The preparation of a pharmaceutical composition that contains at least one composition or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required within the industry.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The composition may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, e.g., Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 μg/kg/body weight, about 5 μg/kg/body weight, about 10 μg/kg/body weight, about 50 μg/kg/body weight, about 100 μg/kg/body weight, about 200 μg/kg/body weight, about 350 μg/kg/body weight, about 500 μg/kg/body weight, about 1 mg/kg/body weight, about 5 mg/kg/body weight, about 10 mg/kg/body weight, about 50 mg/kg/body weight, about 100 mg/kg/body weight, about 200 mg/kg/body weight, about 350 mg/kg/body weight, about 500 mg/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 mg/kg/body weight, etc., can be administered, based on the numbers described above.

### Gene Therapy

Gene therapy refers to the transfer of genetic material (e.g. DNA or RNA) of interest into a host to treat or prevent a genetic or acquired disease or condition phenotype. The genetic material of interest encodes a product (e.g. a protein, polypeptide, peptide, functional RNA, antisense) whose production in vivo is desired. For example, the genetic material of interest can encode a hormone, receptor, enzyme, polypeptide, or peptide of therapeutic value. Alternatively, the genetic material of interest can encode a suicide gene. For a review see, in general, the text “Gene Therapy” (Advances in Pharmacology 40, Academic Press, 1997).

Two basic approaches to gene therapy have evolved: (1) ex vivo and (2) in vivo gene therapy. In ex vivo gene therapy cells are removed from a patient, and while being cultured are treated in vitro. Generally, a functional replacement gene is introduced into the cell via an appropriate gene delivery vehicle/method (transfection, transduction, homologous recombination, etc.) and an expression system as needed and then the modified cells are expanded in culture and returned to the host/patient. These genetically reimplanted cells have been shown to express the transfected genetic material in situ.

In in vivo gene therapy, target cells are not removed from the subject rather the genetic material to be transferred is introduced into the cells of the recipient organism in situ that is within the recipient. In an alternative embodiment, if the host gene is defective, the gene is repaired in situ [Culver, 1998]. These genetically altered cells have been shown to express the transfected genetic material in situ.

The gene expression vehicle is capable of delivery/transfer of heterologous nucleic acid into a host cell. The expression vehicle can include elements to control targeting, expression and transcription of the nucleic acid in a cell selective manner as is known in the art. It should be noted that often the 5′UTR and/or 3′UTR of the gene can be replaced by the 5′UTR and/or 3′UTR of the expression vehicle. Therefore as used herein the expression vehicle can, as needed, not include the 5′UTR and/or 3′UTR of the actual gene to be transferred and only include the specific amino acid coding region.

The expression vehicle can include a promoter for controlling transcription of the heterologous material and can be either a constitutive or inducible promoter to allow selective transcription. Enhancers that can be required to obtain necessary transcription levels can optionally be included. Enhancers are generally any non-translated DNA sequence that works contiguously with the coding sequence (in cis) to change the basal transcription level dictated by the promoter. The expression vehicle can also include a selection gene as described herein below.

Vectors can be introduced into cells or tissues by any one of a variety of known methods within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al (1986) and include, for example, stable or transient transfection, lipofection, electroporation, and infection with recombinant viral vectors. In addition, see U.S. Pat. No. 4,866,042 for vectors involving the central nervous system and also U.S. Pat. Nos. 5,464,764 and 5,487,992 for positive-negative selection methods.

Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of DNA viral vector for introducing and expressing recombinant sequences is the adenovirus-derived vector Adenop53TK. This vector expresses a herpes virus thymidine kinase (TK) gene for either positive or negative selection and an expression cassette for desired recombinant sequences. This vector can be used to infect cells that have an adenovirus receptor that includes most cancers of epithelial origin as well as others. This vector as well as others that exhibit similar desired functions can be used to treat a mixed population of cells and can include for example, an in vitro or ex vivo culture of cells, a tissue or a human subject.

Additional features can be added to the vector to ensure its safety and/or enhance its therapeutic efficacy. Such features include, for example, markers that can be used to negatively select against cells infected with the recombinant virus. An example of such a negative selection marker is the TK gene described above that confers sensitivity to the antibiotic gancyclovir. Negative selection is therefore a means by which infection can be controlled because it provides inducible suicide through the addition of antibiotic. Such protection ensures that if, for example, mutations arise that produce altered forms of the viral vector or recombinant sequence, cellular transformation can not occur.

Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally_This characteristic can be useful if the desired purpose is to introduce a specified gene into only a localized number of targeted cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. The vector(s) to be used in the methods of the invention depends on desired cell type to be targeted and are known to those skilled in the art. For example, if breast cancer were to be treated then a vector specific for such epithelial cells would be used. Likewise, if diseases or pathological conditions of the hematopoietic system were to be treated, then a viral vector that is specific for blood cells and their precursors, preferably for the specific type of hematopoietic cell, would be used.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles that are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed will not contain a genome and therefore cannot proceed through subsequent rounds of infection. The specific type of vector will depend upon the intended application. The actual vectors are also known and readily available within the art or can be constructed by one skilled in the art using well-known methodology.

The recombinant vector can be administered in several ways. If viral vectors are used, for example, the procedure can take advantage of their target specificity and consequently, do not have to be administered locally at the diseased site. However, local administration can provide a quicker and more effective treatment, administration can also be performed by, for example, intravenous or subcutaneous injection into the subject. Injection of the viral vectors into a spinal fluid can also be used as a mode of administration, especially in the case of neuro-degenerative diseases. Following injection, the viral vectors will circulate until they recognize host cells with the appropriate target specificity for infection.

An alternate mode of administration can be by direct inoculation locally at the site of the disease or pathological condition or by inoculation into the vascular system supplying the site with nutrients or into the spinal fluid. Local administration is advantageous because there is no dilution effect and, therefore, a smaller dose is required to achieve expression in a majority of the targeted cells. Additionally, local inoculation can alleviate the targeting requirement required with other forms of administration since a vector can be used that infects all cells in the inoculated area. If expression is desired in only a specific subset of cells within the inoculated area, then promoter and regulatory elements that are specific for the desired subset can be used to accomplish this goal. Such non-targeting vectors can be, for example, viral vectors, viral genome, plasmids, phagemids and the like. Transfection vehicles such as liposomes can also be used to introduce the non-viral vectors described above into recipient cells within the inoculated area. Those of skill in the art know such transfection vehicles.

The invention will be further described in the following examples, which do not limit the scope of the invention described in the claims.

### EXAMPLE 1

### General Methods

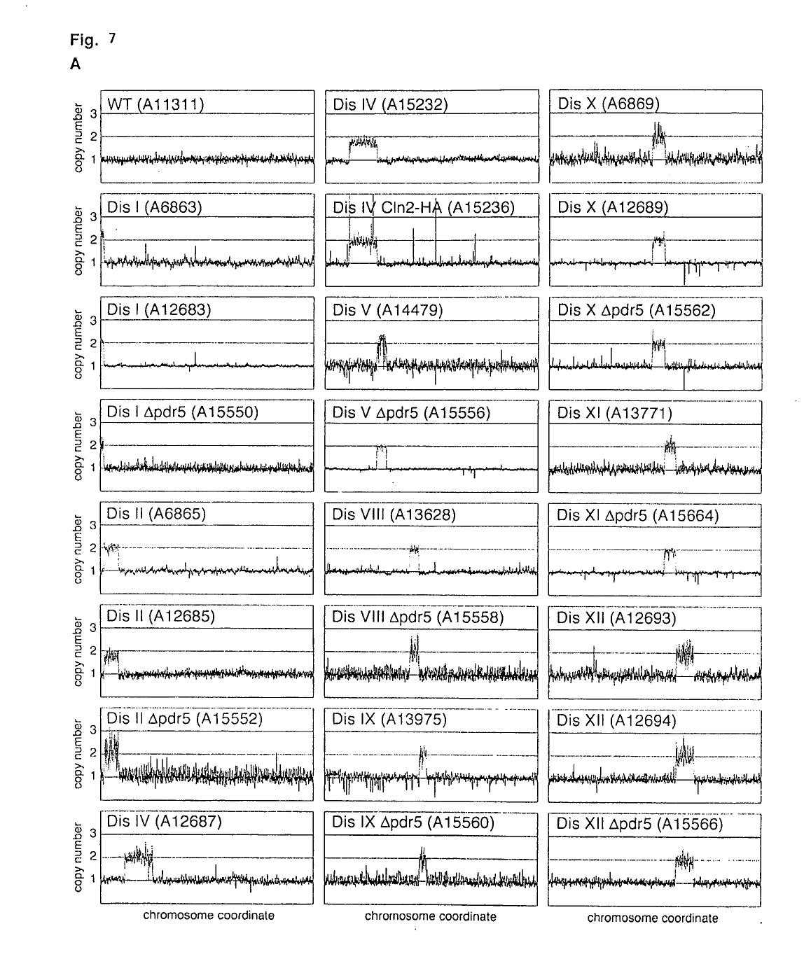

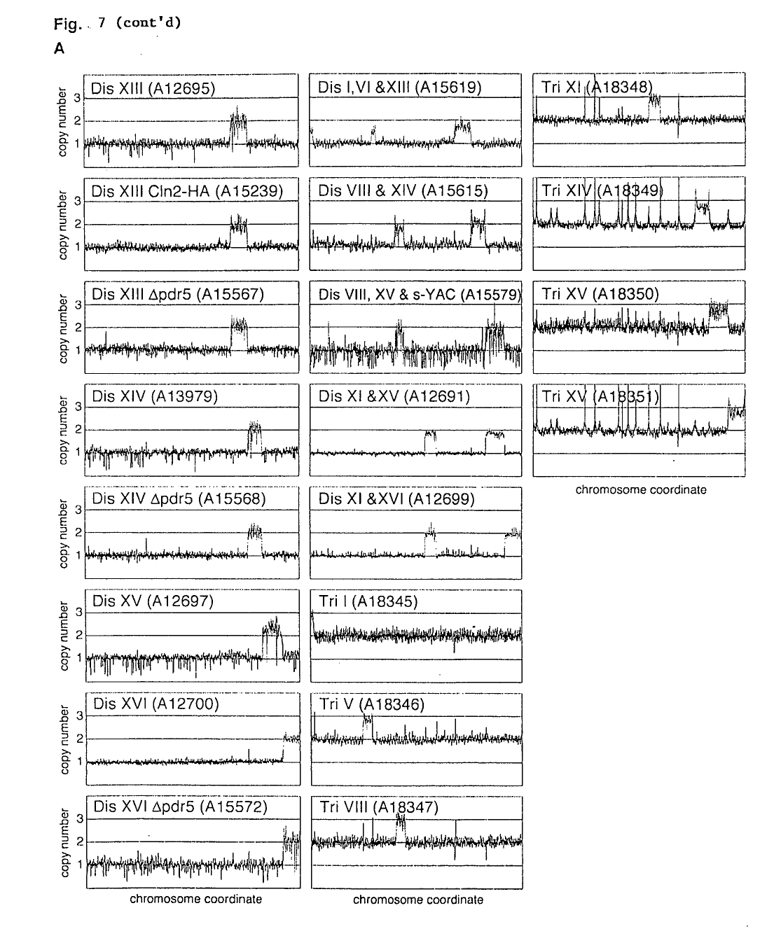

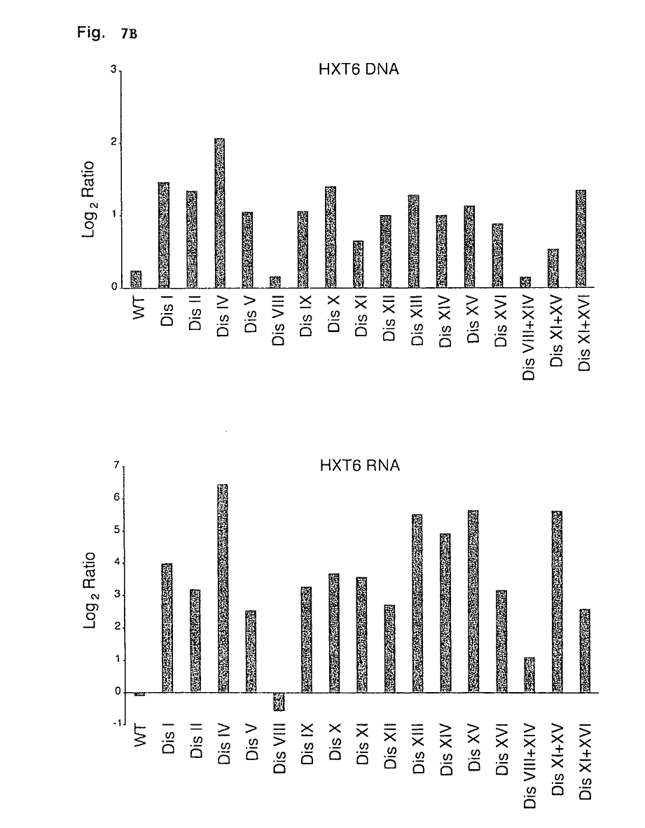

Strains and plasmids. All strains except strain A5644 are derivatives of W303 (A2587) and are listed in Table S2. The CLN2-HA fusion is described in Tyers et a. (S1). Thepdr5:: TRP1 deletion was generated using the PCR-based methods described by Longetine et al. (S2). Aneuploids carrying a deletion of PDR5 or a CLN2-HA fusion were obtained by crossing aneuploid strains to strains carrying the deletion and fusion, respectively. Strains disomic for the chromosome of interest were readily recovered in such crosses. All aneuploid strains used in this study were subjected to comparative genomic hybridization (CGH) to ensure that the additional chromosome was present in its entirety (FIG. 7A). Gene expression arrays were used to show that strains carrying YACs did not contain additional chromosomes.

Generation of aneuploid yeast strains. Strains carrying extra chromosome(s), henceforth referred to as aneuploid strains, were generated by a chromosome transfer strategy described by Hugerat et al. (S3). The strategy is outlined in FIG. 6. A HIS3 cassette is integrated at a particular location on each chromosome using the PCR-based method described by Longetine et al. (S2). The site of HIS3 integration on each chromosome is described in Table S2. The strain is then mated to a strain carrying the kar1 d1 5 allele, which renders the strain defective in karyogamy (STEP 1 in FIG. 6) (S4, S5). In addition the strain carries the cyh2-Q37E allele, which confers resistance to cycloheximide in a recessive manner (S6). 10⁸ cells of each strain were mixed an incubated for 8-10 hours in YEPD medium at room temperature. The mating mixture was then plated on medium lacking histidine and containing 3 [tg/ml cycloheximide to select for the marked chromosome and to select against diploids and heterokaryons. kar1A15 cells carrying the HIS3 marked chromosome were then mated to cells that carried the kanMX6 cassette at the same genomic locus where the HIS3 was integrated (STEP 2 in FIG. 6). This strain also carries the can1-100 allele, which confers resistance to canavinine in a recessive manner. Matings were performed as described above and the mating mixture was plated on medium containing G418 and lacking histidine to select for the presence of the disome. To select against mating events the medium also contained canavinine.

Two copies of the can1-100 allele no longer confer canavinine resistance (data not shown). Because the can1-100 allele was located on chromosome V, strains disomic for chromosome V had to be constructed by a different strategy. Instead of integrating HIS3 and kanMX6 at the same genomic location HIS3 was integrated at the CAN1 locus and kanMX6 was integrated at the intergenic region (187520-187620) between ORFs YERO15 W and YERO16W.

Cells disomic for chromosomes III and VII were not obtained because the MAT locus and the CYH2 locus are located on chromosome III and VII, respectively. Despite several attempts, we failed to obtained strains disomic for chromosome VI. This finding suggests that two copies of chromosome VI are lethal. ACT1 and TUB2, which encode actin and β-tubulin are located in chromosome VI. Cells are extremely sensitive to increased levels of actin or β-tubulin (S7-S9), which could explain our inability to obtain cells disomic for this chromosome. Consistent with this idea is the observation that we obtained strains disomic for chromosomes I, VI and XIII in two independent attempts to isolate strains disomic for chromosome VI. The gene encoding α-tubulin is located on chromosome XIII and increasing the amount of the α-tubulin has been shown to rescue the lethality associated with excess (β-tubulin (S9).

The CGH analysis revealed that several of our disomic candidate strains not only carried the chromosomes selected for but also an extra chromosome. Although we were not able to select for the presence of the additional chromosome, gene expression and CGH analyses showed that the karyotypes of these multiple disomic cells were stable enough to conduct the experiments described in this manuscript.

Generation of trisomic strains. The kanMX6 marker of disomic strains was replaced with URA3. The resulting disomes carrying a HIS3 and URA3 marker at the same genomic location were mated to a haploid containing the kanMX6 marker at the same locus and trisomic strains were recovered by selection on −His-Ura+G418 media.

Generation of strains carrying YACs. All strains containing yeast artificial chromosomes (YACs) were generated by the procedure used to transfer yeast chromosomes except cells were plated on medium lacking uracil to select for the presence of the YAC. kar1Δ15 strains carrying YAC-7 and YAC-1 were described in Huang et al. (S10). YAC-2, -3, -4, -5 and -6 contain regions of the human Y chromosome and were obtained through ATCC (cat#77393) and are described in Foote et al. (S11). The presence of the YAC was confirmed by Pulse Field Gel Electrophoresis and Southern blotting using a probe for URA3 as described in Huang et al. (S10).

CGH analysis of aneuploid strains. To prepare genomic DNA, cells were grown to saturation in selective media. 15 mls of culture were spun down, rinsed and incubated for 60 minutes at 37° C. in 1.5 mls of 1 M Sorbitol, 10 mM Na-phosphate, pH 7.0, 10 mM EDTA, 200 μg/ml zymolase and 150 μM (3-mercaptoethanol. Cells were pelleted and incubated in 1.5 mls of 50 mM EDTA, pH 8.0, 0.3% SDS, 200 μg/ml proteinase K and incubated for another 60 minutes at 65° C. 0.6 mls of 5 M KOAc was added and incubated on ice for 30 minutes. After centrifugation, the supernatant was subjected to a phenol/chloroform extraction and DNA was precipitated. The DNA was RNAse treated at 37° C. for 2 hours (10 mM Tri-HCl, 1 mM EDTA, pH 7.5, 1 mg/ml RNAse), followed by another phenol/chloroform extraction, and precipitated with ethanol.

1 μg HhaI digested DNA was labeled with Klenow polymerase and Cy3- or Cy5-dCTP according to the BioPrime CGH labeling kit (Invitrogen), using half volume reactions. Yield and dye incorporation were checked with a Nanodrop spectrophotometer. 200 ng differentially labeled DNA from the reference strain and the strain of interest were mixed, combined with control targets and hybridization buffer, boiled for 5 minutes, and applied to a microarray consisting of 60 mer probes for each yeast open reading frame (Agilent). Microarrays were rotated at 60° C. for 17 hours in a hybridization oven (Agilent). Arrays were then washed according to the Agilent SSPE wash protocol, and scanned on an Agilent scanner. The image was processed using the default settings with Agilent Feature Extraction software. All data analysis was performed using the resulting log₂ ratio data, and filtered for spots called as significantly over background in at least one channel.

Gene expression arrays. Total RNA was isolated from cells frozen on filters. Filters were incubated for 1 hour at 65° C. in lysis buffer (10 mM EDTA, 0.5% SDS, and 10 mM Tris, pH 7.5) and acid phenol. The aqueous phase was further extracted twice with an equal volume of chloroform using phase lock gel (Eppendorf). Total RNA was then ethanol precipitated and further purified over RNeasy columns (Qiagen). RNA quality was checked using the Bioanalyzer RNA Nano kit, and 325 ng was used for microarray labeling with the Agilent Low RNA Input Fluorescent Linear Amplification Kit. Reactions were performed as directed except using half the recommended reaction volume and one quarter the recommended Cy-CTP amount. Dye incorporation and yield were measured with a Nanodrop spectrophotometer. Equal amounts of differentially labeled control and sample cRNA were combined such that each sample contained at least 2.5 pmol dye. Samples were mixed with control targets, fragmented, combined with hybridization buffer, and hybridized as described above for the CGH analysis.

Data analysis. The Agilent normalization method assumes an average ratio of 1 between experimental strains and the wild type reference. This assumption is not accurate for strains carrying extra chromosomes. All expression data were therefore renormalized to account for the extra chromosome by averaging the log₂ ratios of all genes not contained on the disomic chromosome. This number was then subtracted from all log₂ ratios in order to make the average log₂ ratios zero for genes not on the disomic chromosome. With this correction, genes contained on all disomic chromosomes over several replicate experiments increased in expression by an average of 1.8-fold. Replicate flasks of the wild-type strain were also compared in order to find the extent of noise in gene expression pattern under these conditions. A cutoff of two standard deviations from the mean (changes of 1.8-fold) was used to remove genes for which the experimental noise could explain the variation in gene expression. Genes were included in the further analysis if their expression exceeded this cutoff in at least one experiment in which gene dosage was not increased compared to wild type (3124 genes).

Expression data obtained from cells grown in the chemostat were filtered and renormalized as described for data obtained from exponentially growing cells. A comparison of two independently grown wild-type samples showed better reproducibility than the batch cultures, with half the standard deviation. Using a cutoff of two standard deviations over the mean (a 1.3 fold change), 4963 genes, most of the yeast genome, changed in at least one experiment in which the gene was not contained on a disome.

Hierarchical clustering was performed using the program WCluster (http://function.princeton.edu/WCluster/). WCluster takes both a data table and a weight table to allow individual measurements to be differentially considered by the clustering algorithm. Expression data were clustered by a Pearson correlation metric with equal weighting given to all data, or with no weight given to genes on the disomic chromosomes, as indicated in the text.

Batch culture growth conditions. Wild-type and disomic strains were grown to OD₆₀₀=1 in −His G418 medium.

Chemostat growth conditions. ATR Sixfors fermenters were modified for use as chemostats. Chemostat cultures were run at 30° C. at a working volume of 300 mls, mixed at 400 rpm, and sparged at 5 standard liters per minute with humidified and filter-sterilized air. The dilution rate was set to 0.17 volumes/hour. Cultures were run in phosphate limited minimal defined medium containing the following (per liter): 100 mg calcium chloride, 100 mg sodium chloride, 500 mg magnesium sulfate, 5 g ammonium sulfate, 1 g potassium chloride, 500 μg boric acid, 40 μg copper sulfate, 100 μg potassium iodide, 200 μg ferric chloride, 400 μg manganese sulfate, 200 μg sodium molybdate, 400 μg zinc sulfate, 1 μg biotin, 200 μg calcium pantothenate, 1 μg folic acid, 1 mg inositol, 200 μg niacin, 100 μg p-aminobenzoic acid, 200 μg pyridoxine, 100 μg riboflavin, 200 μg thiamine, 50 mg adenine, 50 mg tryptophan, 20 mg uracil, 100 mg lysine, 20 mg methionine, 100 mg leucine, 100 mg G418, and 5 g glucose.

Chemostats were inoculated with 1 ml overnight culture grown in chemostat media. Cultures were maintained in batch for 24 hours, at which time the media flow was switched on. Cultures were sampled daily for cell density by Coulter count, klett, and absorbance, and were considered to be in steady state when all parameters were the same for two consecutive measurements, which occurred 4 days after inoculation for all cultures. 100 ml of cultures were harvested by vacuum filtration, flash-frozen in liquid nitrogen, and stored at −80° C. until RNA extraction. 50 ml cultures were harvested onto pre-weighed filters, baked overnight, and weighed for yield measurements. Samples of the filtrate were also saved for analysis.

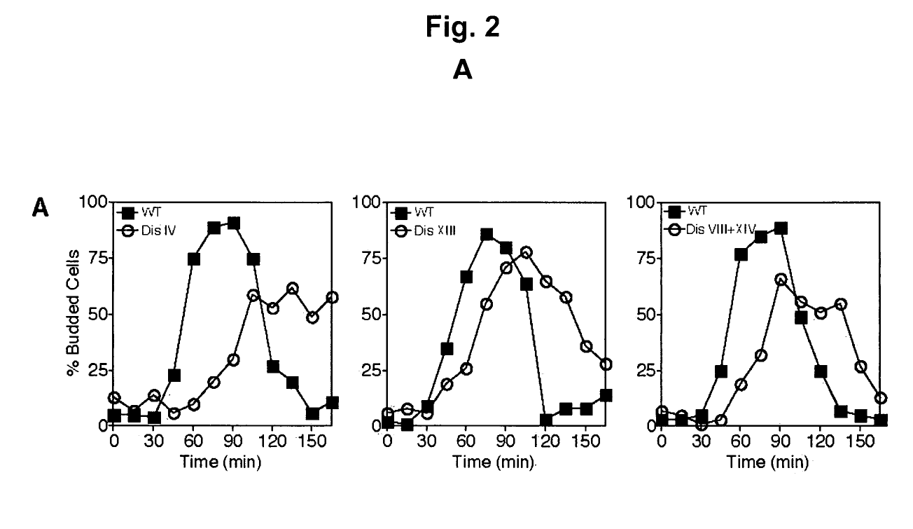

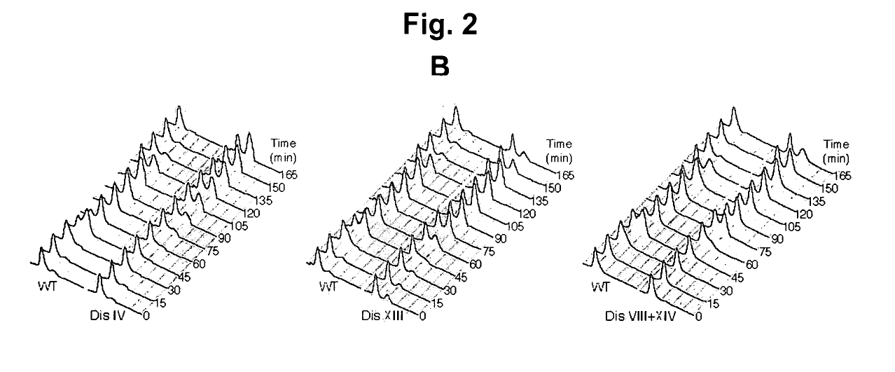

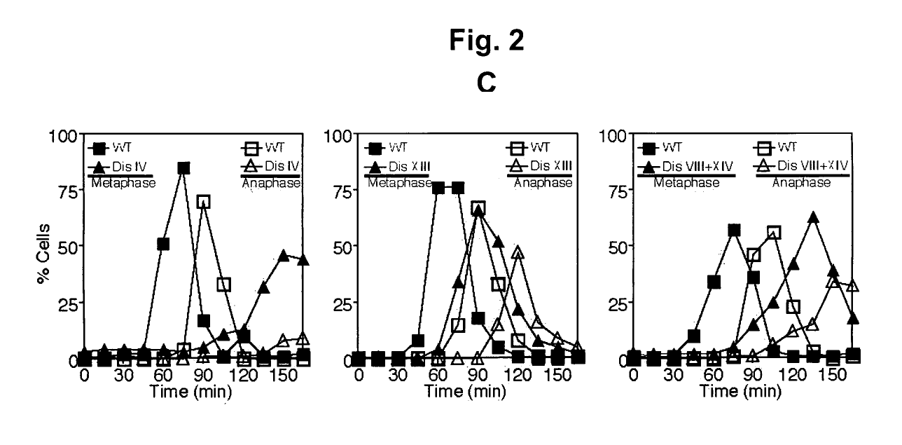

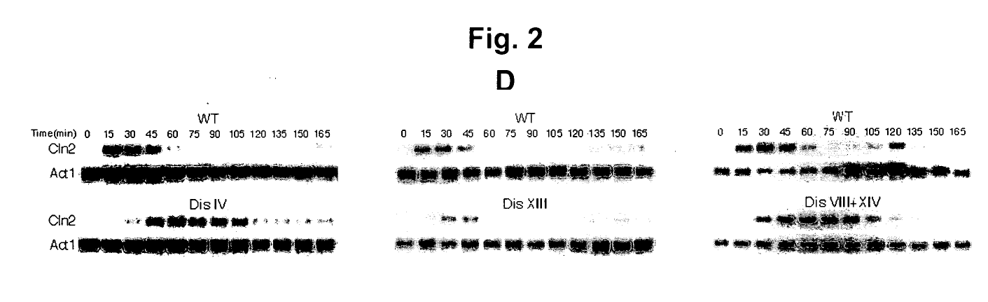

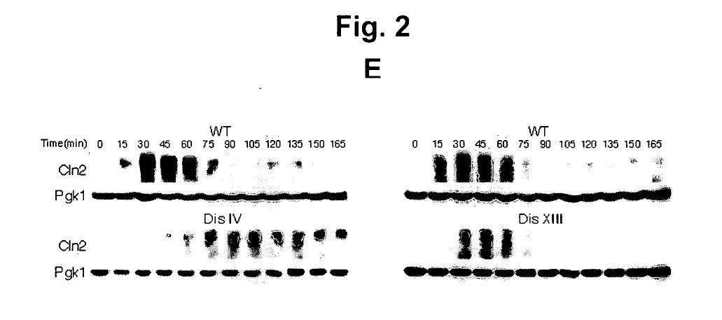

Cell cycle analyses. Cells were arrested in G1 in YPD with 5 μg/ml α-factor for 3 hours. 1.5 hours into the arrest 2.5 μg/ml α-factor was readded. Cells were washed with 10 volumes of YEPD and released into medium lacking pheromone. In the analysis shown in FIG. 2 pheromone (5 μg/ml) was readded when most cells had budded (between 75 and 105 minutes after release from the G1 block, to prevent cells from entering the next cell cycle). Two independent isolates of strains disomic for chromosome I, II, IV, VIII, X, XII and XIII were analyzed revealing highly reproducible cell cycle delays between isolates. Onset of a cell cycle event such as budding or entry into metaphase were determined to occur when 50 percent of the maximal number of cells had executed a particular cell cycle event and were compared to a wild type strain that was analyzed simultaneously.

Cell viability analysis. Cells were grown in selective media overnight and counted using a Multizer 3 Coulter counter. 500 cells were plated on YPD plates and colonies were counted after 2-3 days. Viability is reported as the fraction of the cells plated able to form colonies. In the case of strains disomic for chromosomes IV, XI, XI+XV or I+VI+XIII 2,000-5,000 cells were plated. To determine the viability of cells grown to saturation the number of cells plated was increased as viability decreased. Only data from plates containing 100 colonies or more were included in the analysis.

Quantitative western blot analysis. For quantitative Western blot analysis cells were grown in −His G418 medium to OD₆₀₀=1. 10 mls of culture were harvested and lysed in lysis buffer (50 mM Tris-HCl, pH 7.5, 150 mM NaCl, 2 mM DTT, 2 mM EDTA plus protease inhibitors) using acid washed glass beads. 50, 25, 12.5 and 6.25 μg of lysate were loaded. Nop1 was detected using a mouse anti-Nop 1 antibody at 1:10,000 dilution (Abcam cat#ab4575). Rpa1 was detected using a mouse anti-Rpa1 antibody at 1:1,000 dilution (Genetex cat# GTX16850). Mre11 was detected using a rabbit anti-Mre11 antibody at 1:1,000 dilution (Abcam cat#ab12159). Rps2/Rp132 were detected using a rabbit anti-Rps2 antibody at 1:2,000 dilution (kindly provided by Dr. Jonathan R. Warner). Arp5 was detected using a rabbit anti-Arp5 antibody at 1:1,000 dilution (Abcam cat#ab12099). Tcp1 was detected using a rat anti-Tcp1 antibody at 1:5,000 dilution (Abcam cat#ab2797). Cdc28 was detected using a rabbit anti-Cdc28 antibody at 1:1,000 dilution (S12). Pgk1 was detected using a mouse anti-Pgk1 antibody at 1:25,000 dilution (Molecular Probes cat#A-6457), Pup3 was detected using a mouse anti-Pup3 antibody at 1:1000 dilution (Abcam cat#ab22672). Pre6 was detected using a mouse anti-Pre6 antibody at 1:1,000 dilution (Abcam cat#ab22667). Hht1 was detected using a mouse anti-Hht1 antibody at 1:20,000 dilution (Upstate cat#07-690). Rpt1 was detected using a mouse anti-Rpt1 antibody at 1:5,000 dilution (Abcam cat#ab22678), Lcb4 was detected using a goat anti-Lcb4 antibody at 1:200 dilution (Santa Cruz cat# sc-27723). Elp3 was detected using a goat anti-Elp3 antibody at 1:1,000 dilution (Santa Cruz cat# sc-26320). Eaf3 was detected using a rabbit anti-Eaf3 antibody at 1:1,000 dilution (Abcam cat#ab4467). Yaf9 was detected using a rabbit anti-Yaf9 antibody at 1:1,000 dilution (Abcam cat#ab4468). Fcy1 was detected using a sheep anti-Fcy1 antibody at 1:1,000 (AbD Serotec cat#2485-4906).

Other techniques. CLN2 RNA, Cln2 protein levels and DNA content were analyzed as described in (S13-S15). Glucose measurements were performed using the Glucose Assay kit (Sigma Cat#GAHK-20) according to the manufacture's instructions.

### EXAMPLE 2

### Generation of Aneuploid Yeast Strains

To generate yeast cells that contain an additional chromosome, a chromosome transfer strategy was used. During mating, if one of the mating partners lacks the karyogamy gene KAR1, nuclear fusion does not occur (8). However, occasionally individual chromosomes are transferred from one nucleus to the other during these abortive matings (8, 9). When the two mating partners carry different selectable markers at the same genomic location, these rare chromosome transfers can be selected for (FIG. 6). Using this technique, 13 out of the 16 possible disomic strains were generated (Table 1 and 2, (10)).

To ensure that strains with the correct marker combination were indeed disomic for the entire chromosome, comparative genomic hybridization (CGH) was performed, which allows for the quantification of gene copy number on a genome-wide scale. This analysis also revealed that some of the strains obtained from the chromosome transfer procedure carried one or two extra chromosomes in addition to the one that was selected for (FIG. 7A). Although the second chromosome cannot be selected for, these strains were karyotypically stable enough to conduct a phenotypic characterization.

### EXAMPLE 3

### Aneuploidy Causes a Transcriptional Response

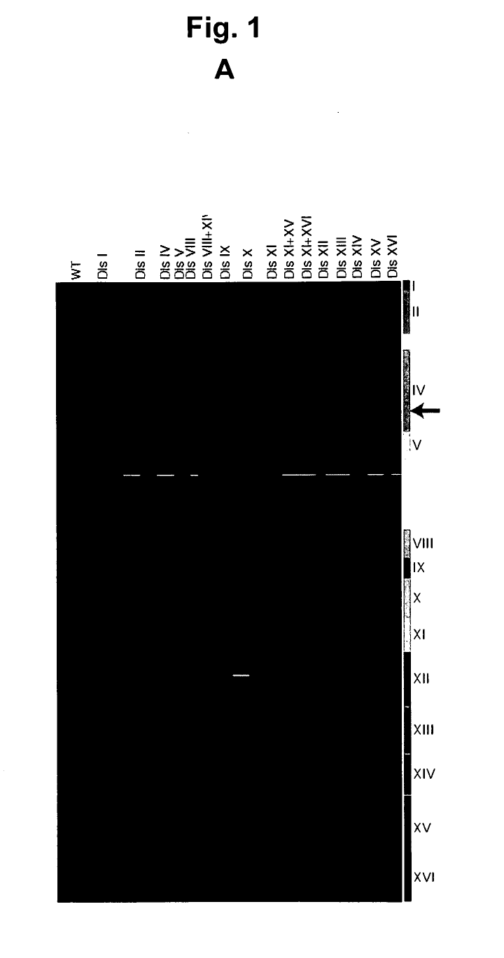

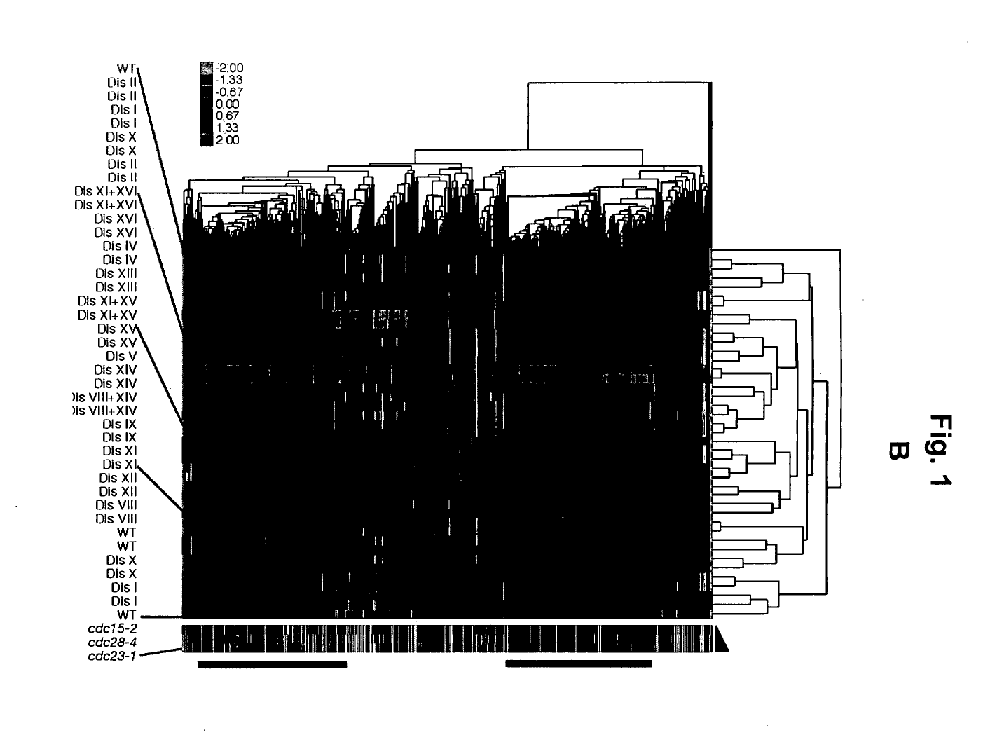

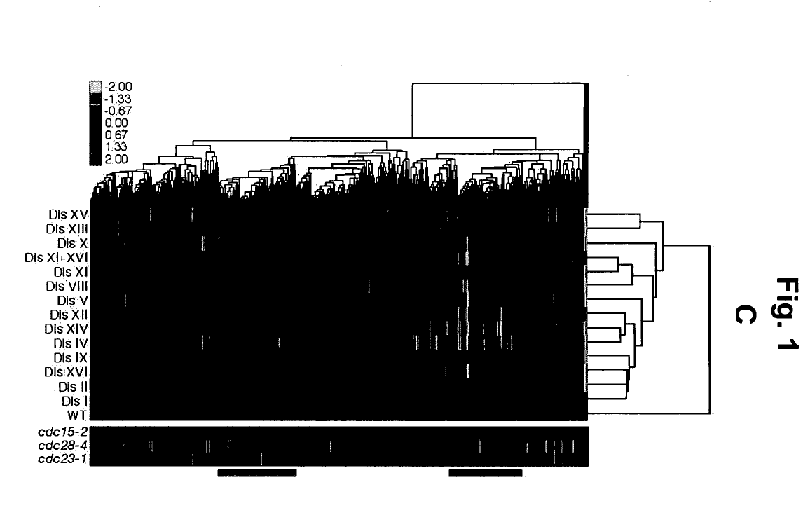

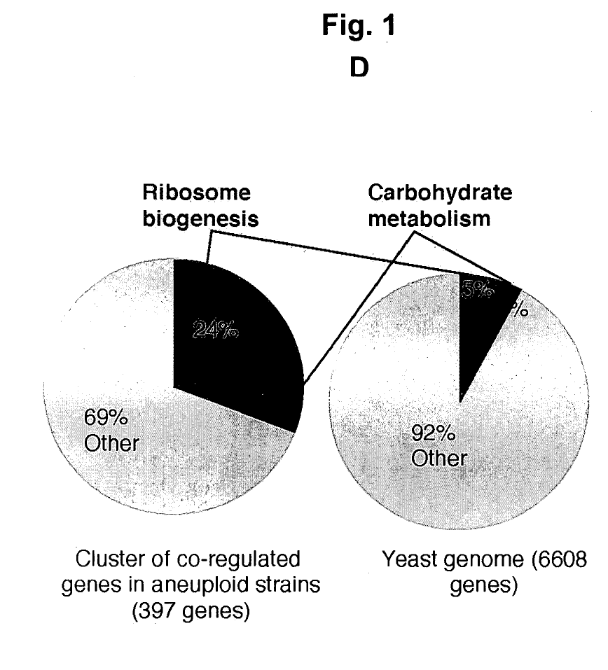

In order to characterize the effects of aneuploidy on gene expression, each aneuploid yeast strain was grown to mid-log phase in batch culture and genome wide gene expression relative to the wild type strain was measured by DNA microarrays. An approximate 2-fold increase in gene expression was observed along the entire length of the disomic chromosomes, indicating that most if not all genes are expressed proportionally to the number of DNA copies in the cell (FIG. 1A). A similar result has been reported for a smaller dataset (11).

To reveal more subtle correlations masked by the strong chromosome-specific signals (FIG. S3A), a clustering program was applied that allows the assignment of a reduced weight to genes on disomic chromosomes (10)(FIG. 1B). This analysis showed that many aneuploid yeast strains, particularly strains disomic for chromosomes IV, XIII, XV, XVI and strains with multiple extra chromosomes exhibited a gene expression signature characteristic of the yeast environmental stress response (ESR). 615 of 870 genes identified by Gasch et al. to constitute the ESR cluster also showed the same transcriptional change in yeast strains with additional chromosomes (FIG. 1B) (12). These same expression changes are also observed in yeast strains growing at slower growth rates (13). Mutants defective in cell proliferation, such as temperature sensitive cdc28-4 or cdc23-1 grown at the permissive temperature (cdc28-4 mutants exhibit a G1 delay, cdc23-1 mutants a metaphase delay) also exhibited some of the same changes in gene expression (FIG. 1B), raising the possibility that defects in cell proliferation could also cause this transcriptional response.

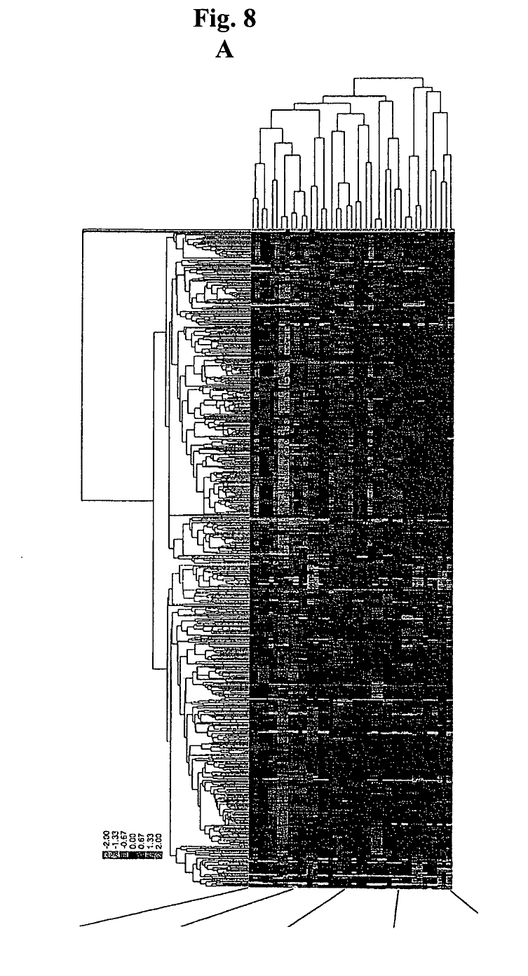

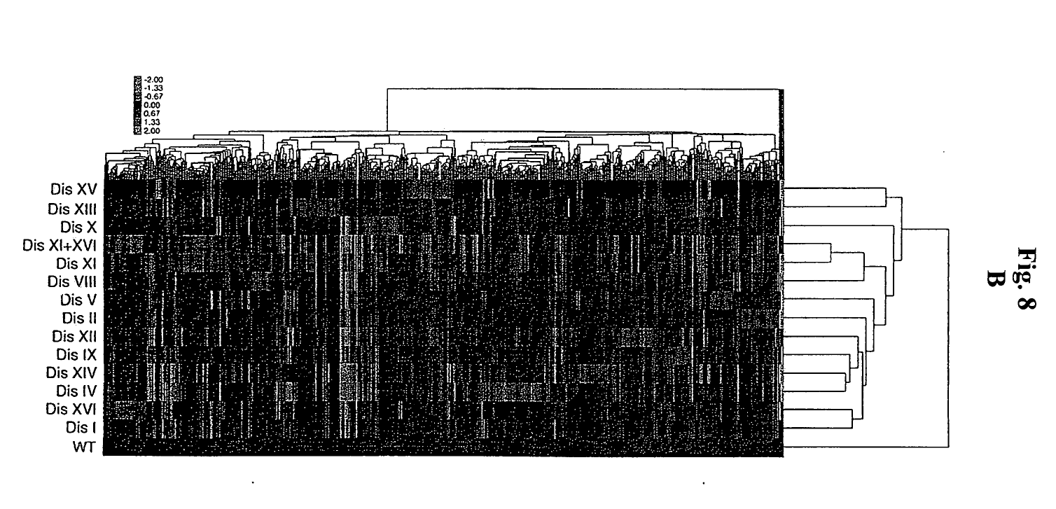

All aneuploid strains that were examined proliferated more slowly than did wild type cells (FIGS. 9, A, B, F and G). Gene expression patterns that are linked to growth rates could thus mask gene expression patterns common to all aneuploid strains. To eliminate differences in gene expression caused by differences in doubling time, all aneuploid strains and the wild type were grown at the same growth rate in the chemostat under conditions where phosphate was limiting. Because the set doubling time of ˜6 hours was longer than the doubling time of each strain in batch growth, all strains grew at the same rate. When cells reached steady state, samples for gene expression were harvested for microarray analysis. Slow-growing strains carrying the cdc28-4 and cdc23-1 mutations were also grown under the same conditions. The gene expression changes that correlated with growth rate differences were not present in any of the chemostat-grown samples. The remaining gene expression changes included a transcription pattern shared by most of the aneuploid strains and not detectable or not present in exponentially growing cultures or in wild-type, cdc28-4 and cdc23-1 mutants grown in the chemostat under phosphate limiting conditions (FIGS. 1C and 8B). Of the 4963 genes whose expression changed greater than the control threshold of 1.3-fold in at least one strain, 397 genes showed changed expression in 10 or more of the 14 aneuploid strains. The program GO Term Finder, available from the Saccharomyces Genome Database (14) was used, to identify the functional categories enriched in each gene set. The group that showed increased expression was enriched in ribosomal biogenesis genes, particularly those related to rRNA processing (FIG. 1D and Table 4). Genes with annotations related to nucleic acid metabolism were also enriched (Table S4). The more variable set of genes whose expression was decreased was enriched for genes involved in carbohydrate metabolism (FIG. 1D and Table 4). These results indicate that aneuploid strains, when normalized for growth rate in phosphate-limited chemostats, are somehow perturbed with respect to ribosomal biogenesis and energy production.

### EXAMPLE 4

### Aneuploid Yeast Strains Exhibit a G1 Delay

To determine how aneuploidy affects cell physiology, the proliferation properties of strains carrying one or several extra chromosomes were characterized. The doubling time and cell size was slightly increased in most aneuploid strains in complete medium (YEPD; FIG. 9A) and synthetic medium that selects for the presence of the disome (−His+G418; FIG. 9B). Even disomic strains that did not exhibit a proliferation delay such as cells disomic for chromosome I or II showed decreased proliferative capacity compared to wild-type cells when the strains were co-cultured (FIGS. 9, F and G). Furthermore, some of the aneuploids, such as strains disomic for chromosome IV, XI or XIII also exhibited poor viability as judged by their inability to form colonies on plates (FIG. 9E). The proliferative disadvantage and increase in cell size was also observed in diploid cells carrying an extra chromosome (FIGS. 9, C and D) indicating that the gain of an extra chromosome interferes with cell proliferation of both haploid and diploid cells. Thus, contrary to what we would have expected from studies on cancer cells, where aneuploidy is thought to bring about a proliferative advantage (4), aneuploidy causes a proliferative disadvantage in yeast.

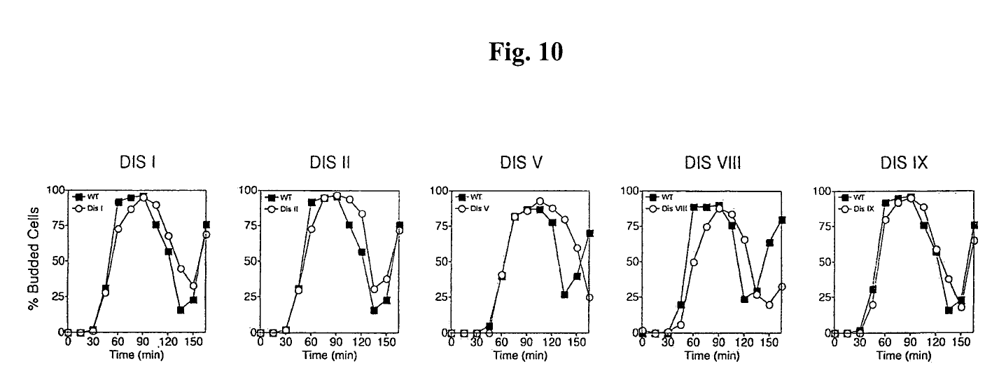

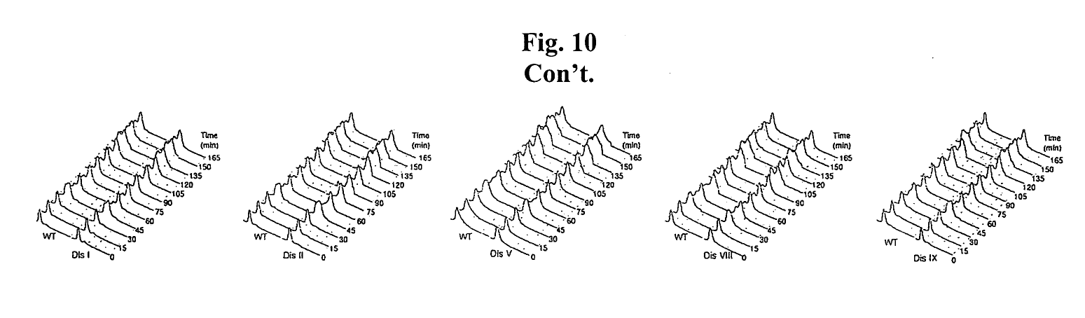

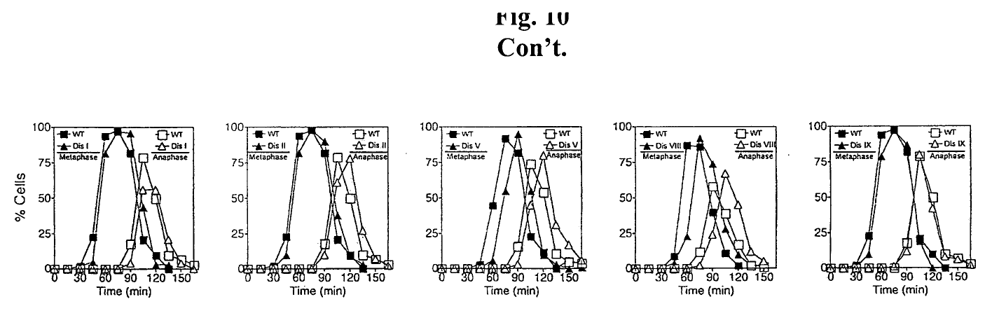

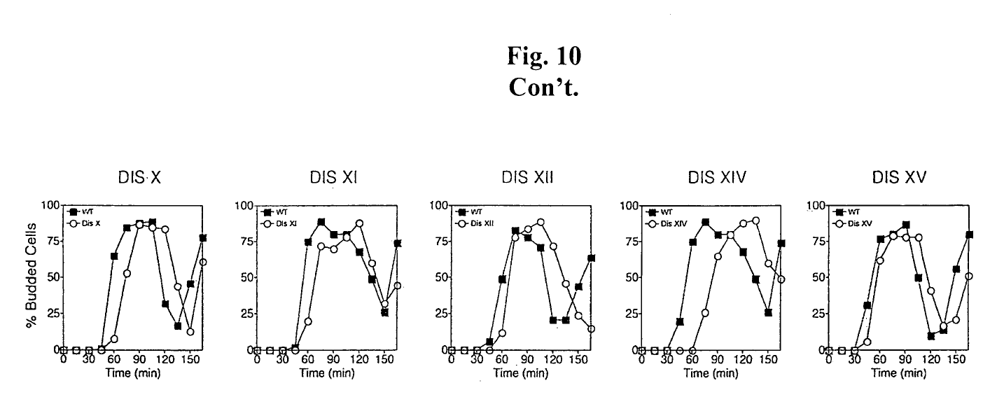

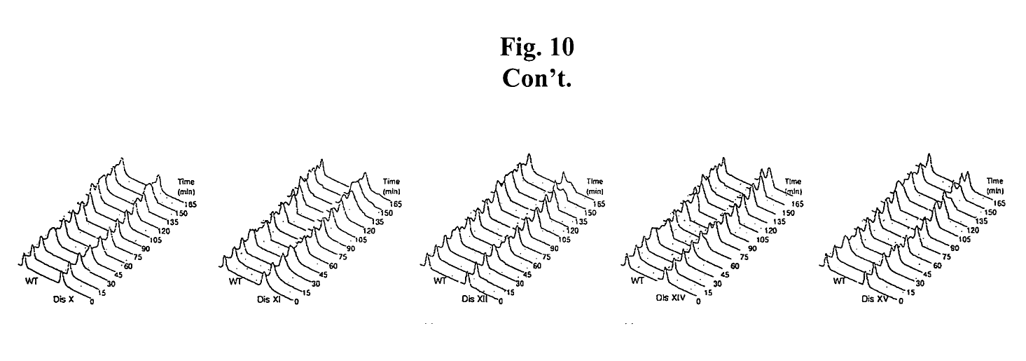

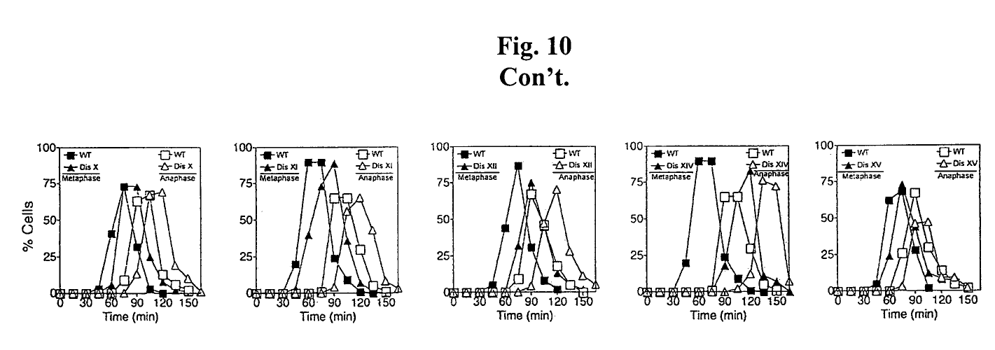

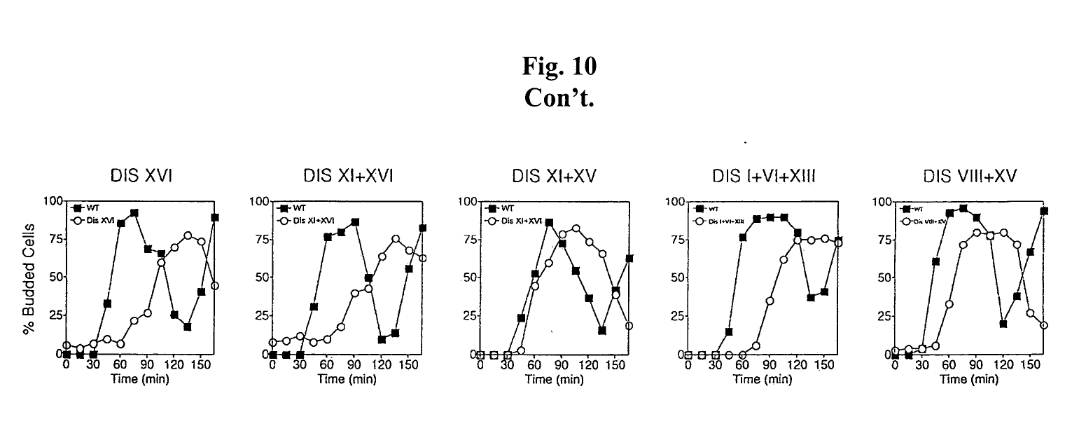

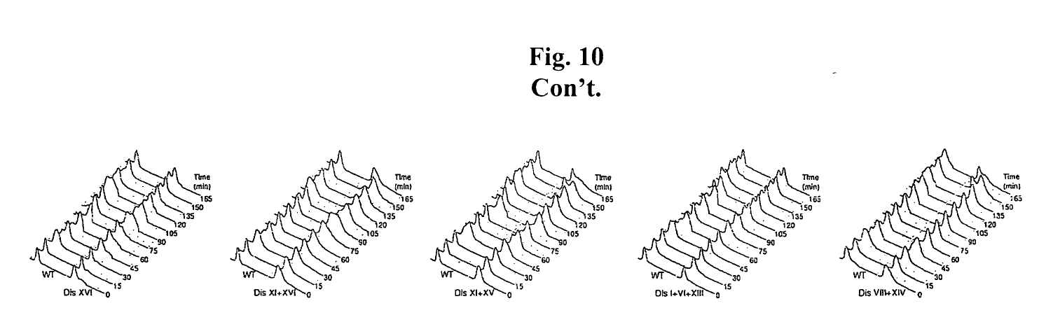

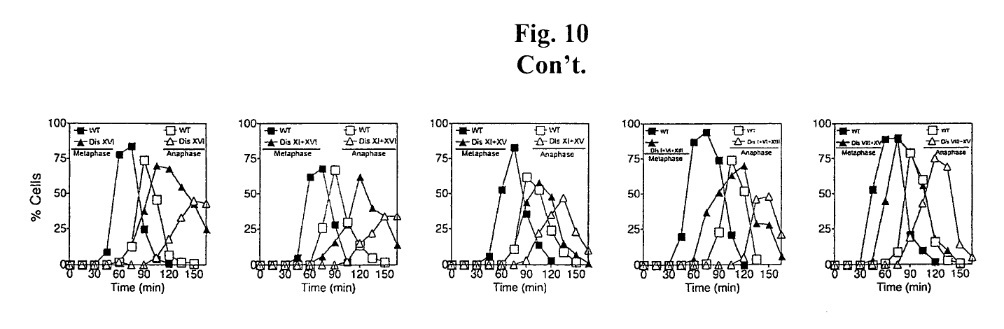

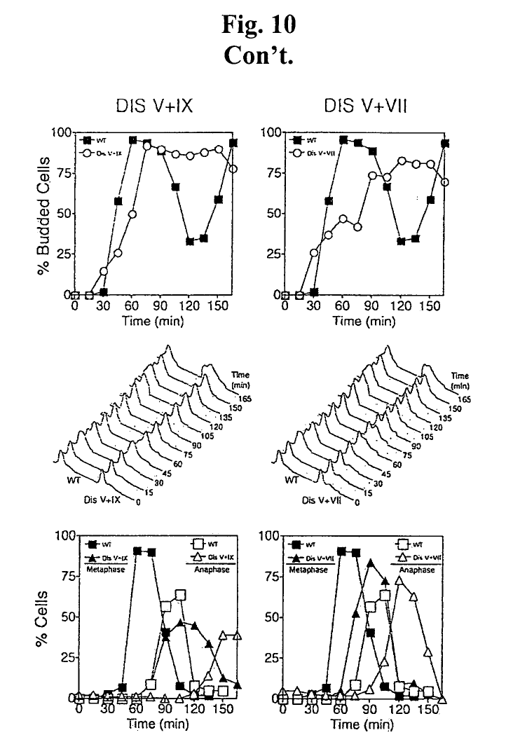

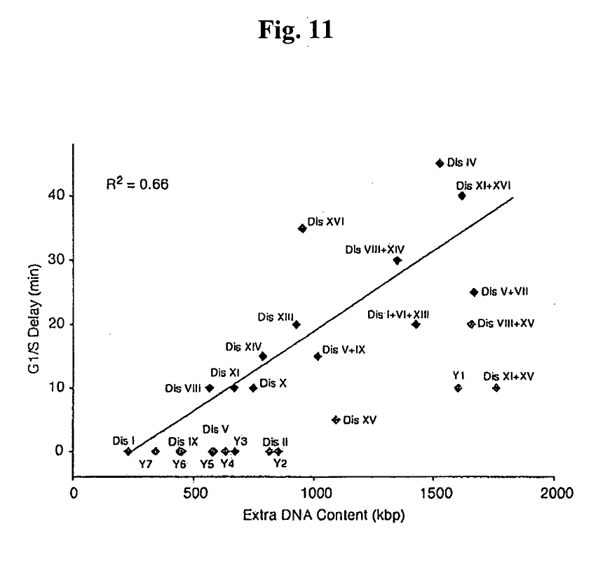

To determine in which stage of the cell cycle the aneuploid yeast strains were delayed, cell cycle progression after release from a pheromone-induced G1 phase arrest was examined. Entry into the cell cycle, as judged by bud formation (FIG. 2A) and DNA replication (FIG. 2B) was delayed in 16 of 20 aneuploid strains. With the exception of cells disomic for chromosome I, II, V, or IX, all aneuploid strains exhibited a delay in entry into the cell cycle (FIG. 10 and Table 1) with most strains (disome VIII, X, XI, XII, XIII, XIV, XVI, V+IX, VIII+XV and XI+XV strains) showing a delay ranging from 10 to 20 minutes. Cells disomic for multiple chromosomes (disome V+VII, VIII+XIV, XI+XVI and I+VI+XIII strains) as well as cells disomic for chromosome IV or XVI exhibited a G1 delay of 25 minutes or more. Aneuploids exhibited few other cell cycle delays. The metaphase to anaphase transition was delayed in only 2 of the 20 aneuploid strains (FIG. 10 and Table 1) and only 6 out of 20 exhibited a delay in entry into mitosis (as determined by a delayed appearance of cells with metaphase spindles, FIG. 2C, Table 1 and FIG. 10). These results indicate that most aneuploid strains are delayed in G1 phase. In general, the delay appears to be larger in strains carrying an extra copy of a large chromosome or extra copies of multiple chromosomes (FIG. 116) suggesting that the amount of additional yeast DNA may contribute to determining the length of the G1 delay.

The molecular events underlying the G1 to S phase transition are well characterized in S. cerevisiae. The cyclin-dependent kinase (CDK) Cdc28 associated with the cyclin Cln3, inhibits Whi5, an inhibitor of the transcription factor complex SBF (15, 16). SBF in turn induces the transcription of genes encoding two other cyclins CLN1 and CLN2, which when complexed with Cdc28, promote entry into the cell cycle (17). The abundance of CLN2 RNA and Cln2 protein in strains disomic for chromosome IV, XIII or VIII+XIV was analyzed. Accumulation of CLN2 RNA and Cln2 protein was delayed and paralleled the delay in bud formation and DNA replication (FIGS. 2, D and E). These results indicate that in the strains that were analyzed, aneuploidy interferes with the G1 to S phase transition upstream of CLN2 transcription.

### EXAMPLE 5

### Aneuploids Exhibit Increased Glucose Uptake

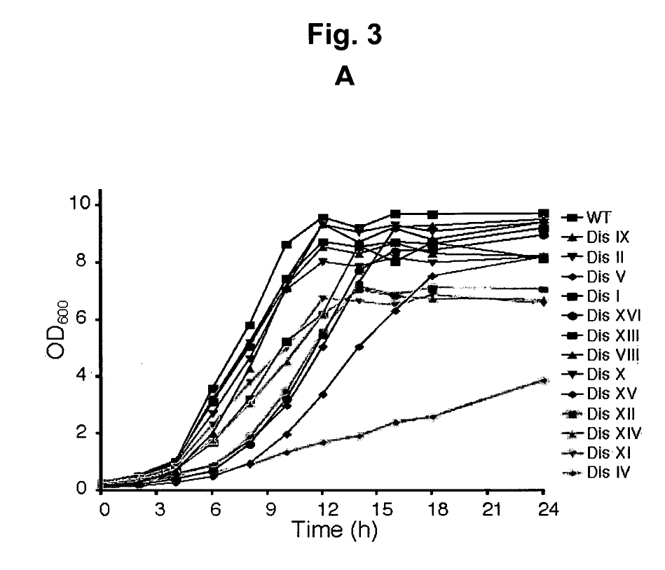

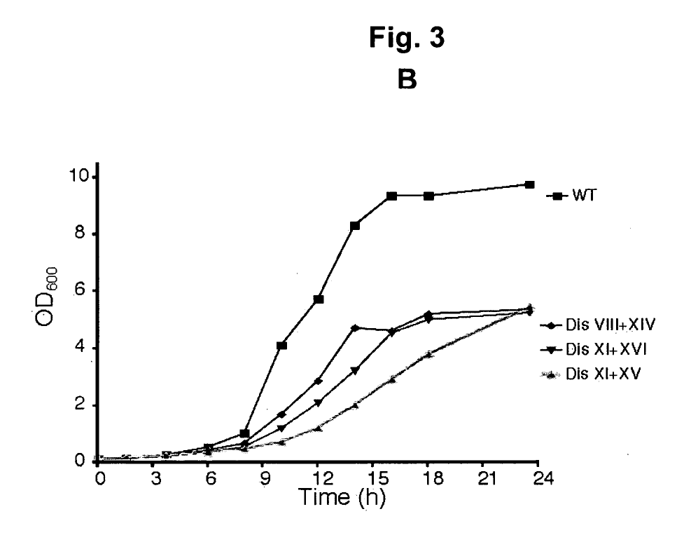

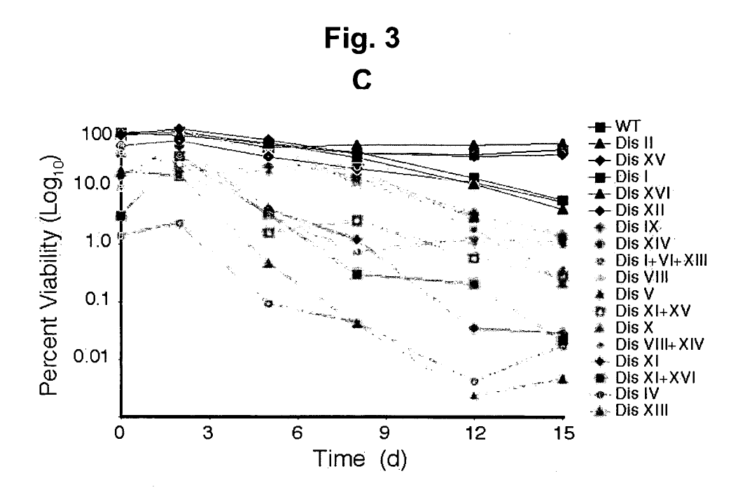

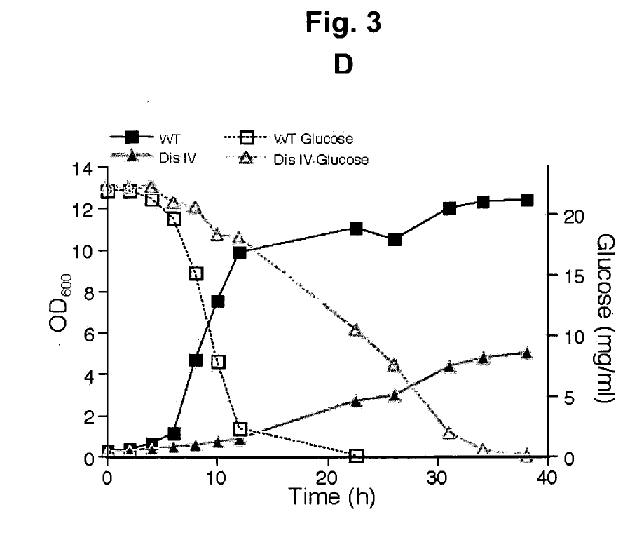

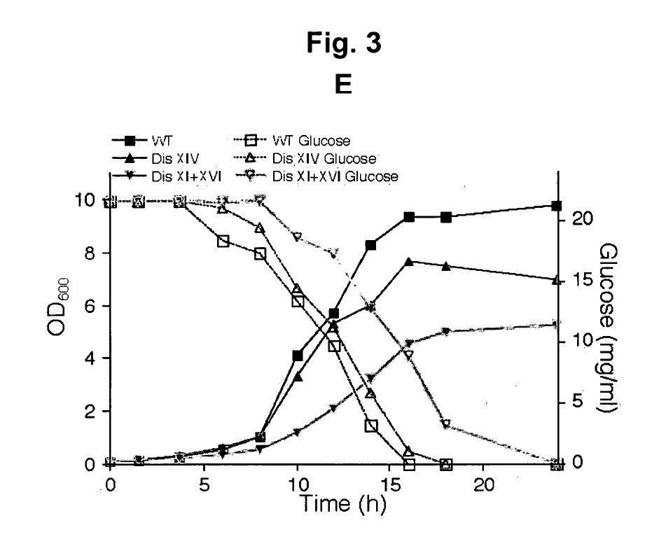

To further investigate the effects of aneuploidy on cell proliferation, the kinetics with which aneuploid cells enter stationary phase were examined. Most aneuploids reached saturation at a lower population size (measured by optical density at 600 nm [OD₆₀₀], FIGS. 3, A and B) and lost viability upon prolonged culturing in stationary phase (FIG. 3C). In general, the maximum OD₆₀₀ was lower in strains carrying two copies of large chromosomes or two copies of multiple chromosomes (FIGS. 3, A and B). Thus, biomass accumulation appears to be inversely correlated with the amount of additional yeast DNA present in the aneuploid strains and the severity of their proliferation defects.

To determine whether the lower OD₆₀₀ at which aneuploids enter stationary phase was due to nutrient depletion, we simultaneously measured glucose uptake and accumulation of biomass. This comparison revealed that wild-type cells generated more biomass per internalized glucose molecule than did aneuploid cells. Whereas wild-type cells reached cell densities of OD₆₀₀=9 having taken up ¾ of the glucose in the medium, cells disomic for chromosome IV only reached a cell density of OD₆₀₀ of less than 4 (FIG. 3D). The increase in glucose uptake correlated with the severity of the cell cycle delay, with strains with a shorter doubling time accumulating more biomass per glucose molecule (FIG. 3E).

Consistent with the idea that aneuploids take up more glucose was the observation that the gene loci encoding the high affinity glucose transporters Hxt6 and Hxt7 were amplified (FIG. S2B) and more highly expressed (FIGS. 1 and 7B) in most of the aneuploid strains we generated (n=42). Strains that did not show this amplification and increased expression were strains carrying an extra copy of chromosome VIII, which carries three genes encoding other high affinity glucose transporters. Together with the microarray experiments indicating changes in gene expression relating to carbohydrate metabolism, these results suggest that aneuploids require more carbohydrates or energy or both for cell survival and proliferation than do wild-type cells.

### EXAMPLE 6

### Most Genes on the Aneuploids'Extra Chromosomes are Expressed

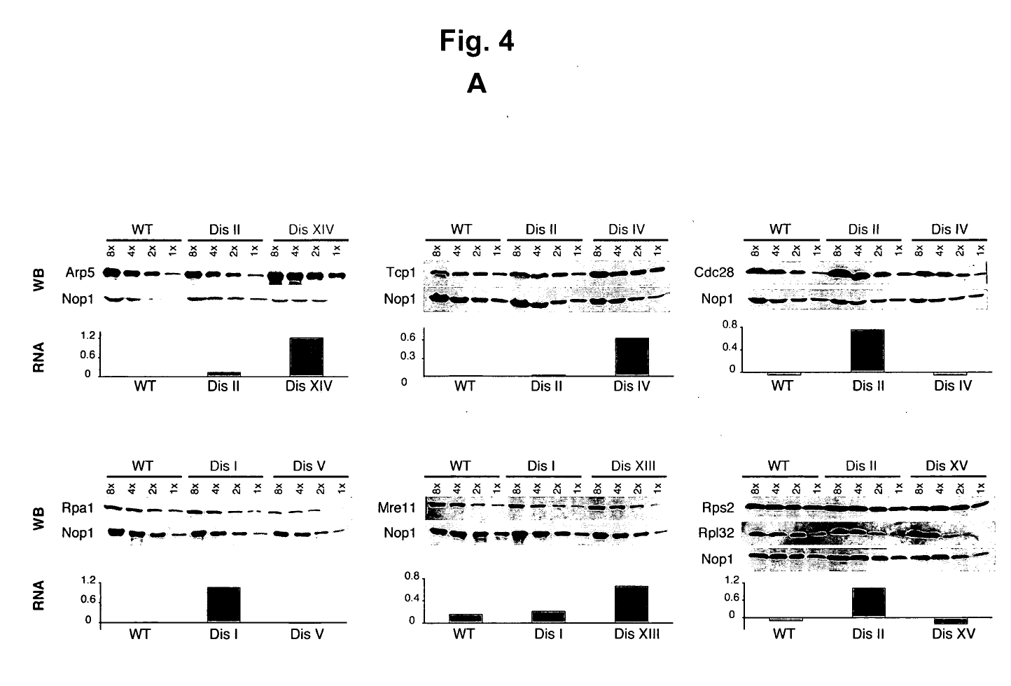

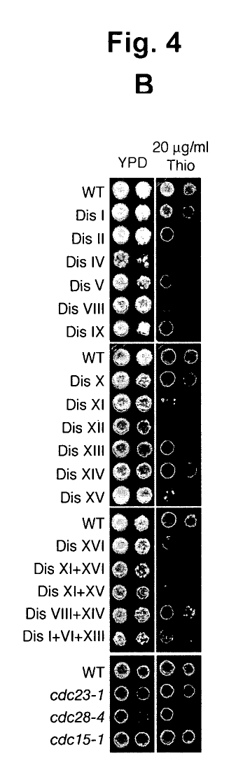

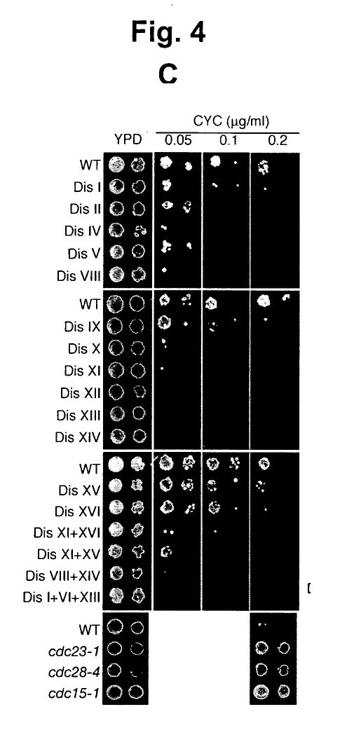

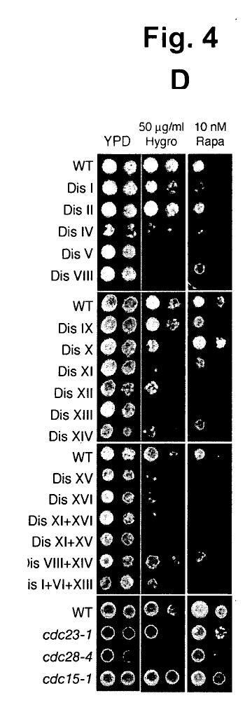

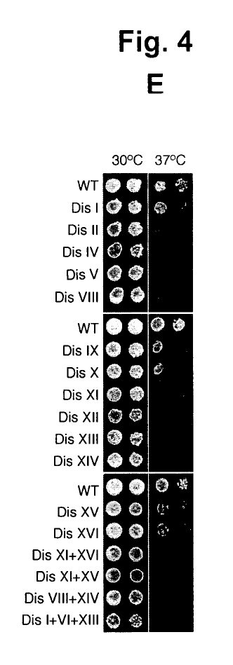

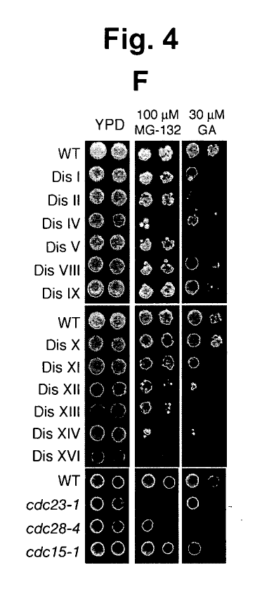

It was hypothesized that macromolecule biosynthesis from the additional chromosome present in aneuploid strains could be one reason why aneuploids need additional glucose. To test this hypothesis expression profile analysis of aneuploids was performed and showed that most genes present on the additional chromosomes were transcribed: 93% of genes carried on the chromosome that was present in two copies were overexpressed at least 1.3-fold over wild type, and expression of 83% of genes went up by 1.5-fold or more (FIGS. 1, A and C). To determine whether the transcripts produced from the extra chromosomes were also translated, the amounts of a small number of proteins were measured. The amounts of Arp5, Tcp 1 and Cdc28 protein were increased in strains disomic for the chromosomes containing the genes encoding these proteins (FIG. 4A). These results suggest that at least some of the genes present on the additional chromosomes are not only transcribed but also translated.

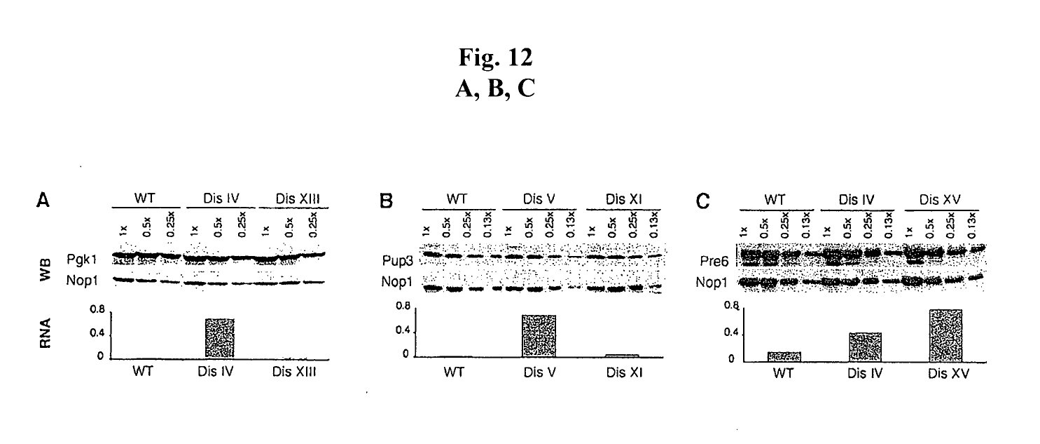

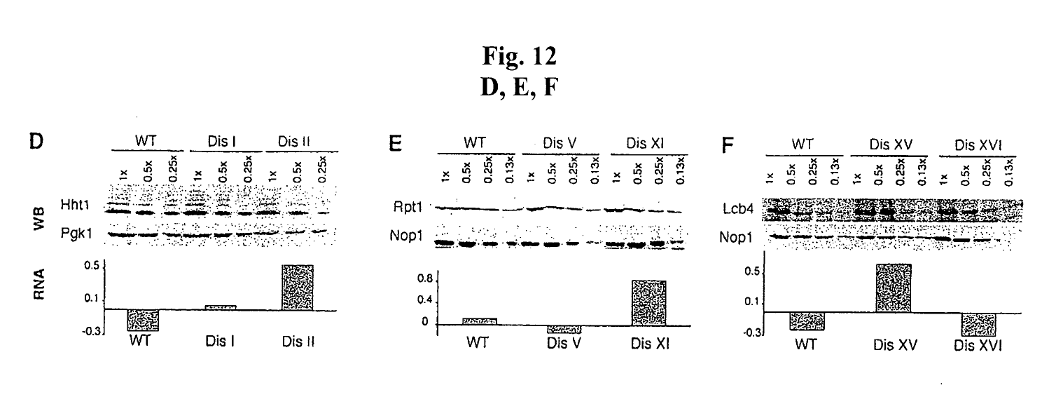

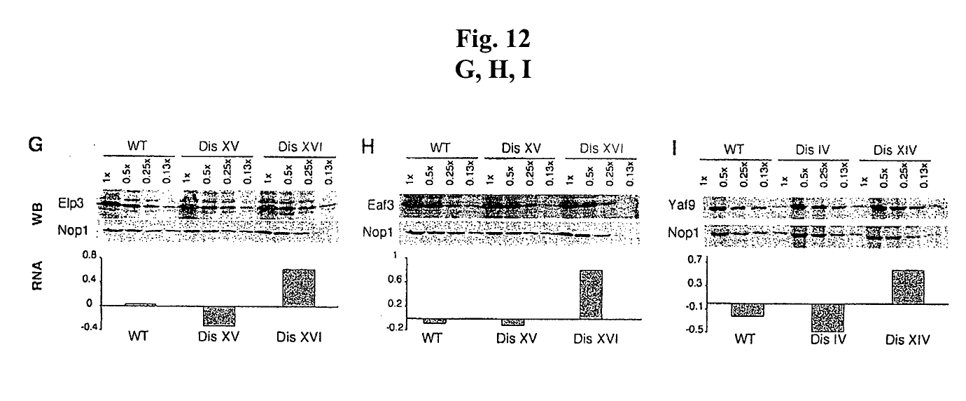

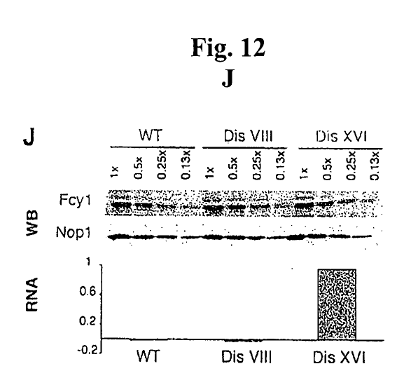

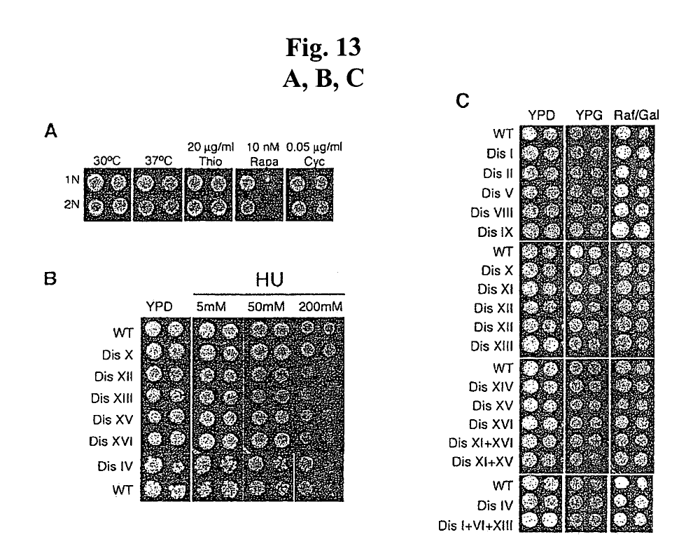

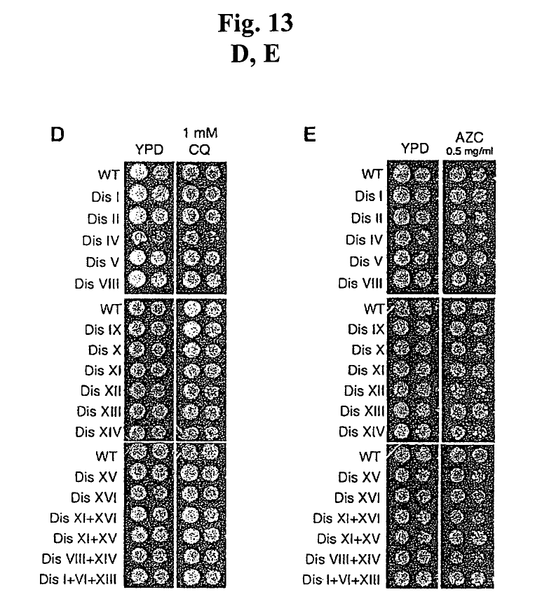

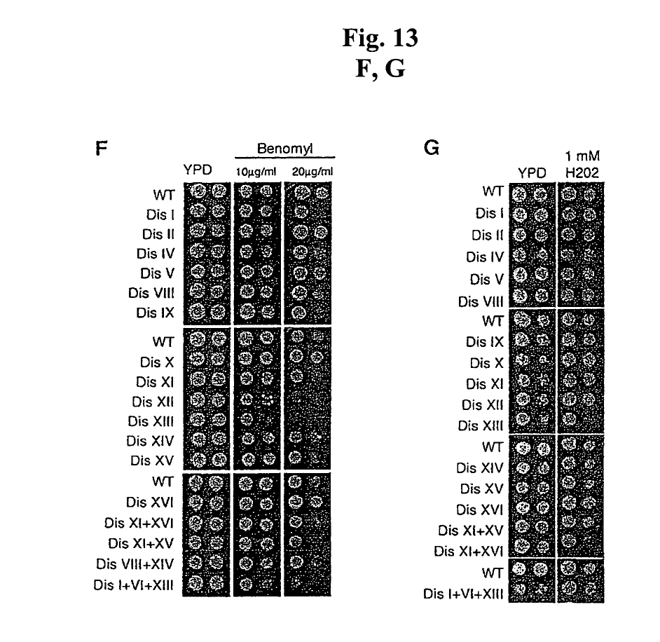

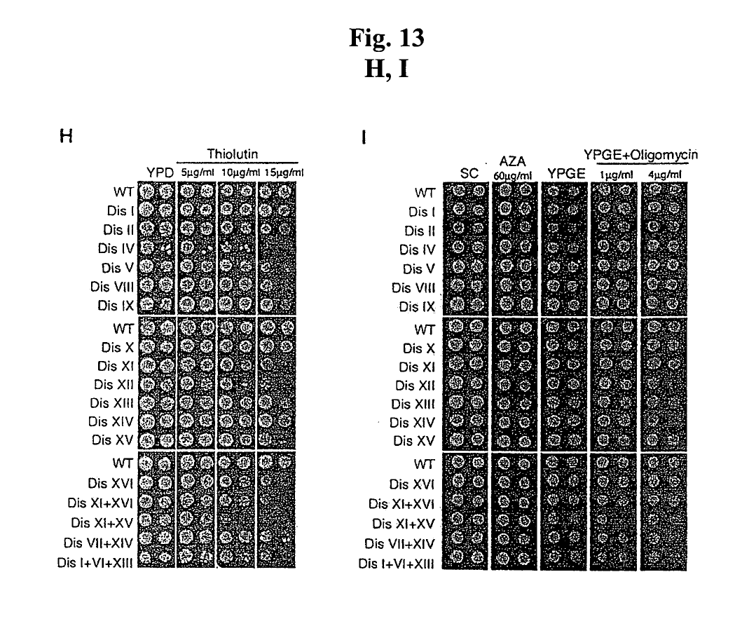

Interestingly, most of the proteins (13 out of 16) that were analyzed showed no change in abundance even though the amount of transcript was increased in accordance with the increase in gene copy number (FIGS. 4A and 12). With the exception of Lcb4 and Fcy1 (for which it is not known whether they are components of protein complexes), all 13 proteins analyzed are components of protein complexes. Rpa1 is a component of the replication factor, Mre1 1 of the RMX complex, Rps2 and Rp132 of the ribosome, Rpt1 of the proteasome, Nop1 of the nucleolus, Histone H3 of the nucleosome, Yaf9 and Eaf3 of the NuA4 histone H4 acetyltransferase complex, and Elp3 of the elongator complex. These findings indicate that many proteins synthesized from the additional chromosomes are either not translated or, more likely, degraded shortly upon synthesis (21).

Consistent with the idea that increased protein degradation occurs in aneuploid yeast strains is the observation that proliferation of a number of aneuploid strains (IV, XII, XIII, XIV, and XVI) is inhibited by concentrations of the proteasome inhibitor MG 132 that wild-type cells grow at as judged by their ability to form colonies on plates containing the drug (FIG. 4F; Note that strains with multiple additional chromosomes could not be tested due to the need of deleting PDR5 to test the effects of MG 132) (22, 23). Furthermore, proliferation of all aneuploid strains was hampered by the protein synthesis inhibitor cycloheximide (FIG. 4C), which can be a sign of ubiquitin-depletion (24). Several proteins such as a-tubulin and histones, which are components of multi-protein complexes are degraded if they are overexpressed or their binding partners are missing (25, 26). Such a mechanism might regulate the amounts of the proteins that did not increase in abundance in accordance with gene dosage in the aneuploid strains. Thus, transcription, translation and degradation of proteins produced from the additional chromosomes present in aneuploids may contribute to the increased glucose uptake of these cells.

### EXAMPLE 7

### Proliferation of Aneuploids is Inhibited by Protein Synthesis Inhibitors and High Temperature

To determine whether the synthesis of proteins from the additional chromosomes and their presence in the cell represents an increased burden on the cell's protein production machinery, the ability of aneuploid strains to grow under conditions that interfere with transcription, protein synthesis and protein folding was examined. Proliferation of all aneuploids with the exception of strains disomic for chromosome I, X, or XIV was inhibited by high (20 μg/ml) concentration of the RNA polymerase inhibitor Thiolutin (FIG. 4B). At low concentrations of the RNA polymerase inhibitor (5 μg/ml to 15 μg/ml) proliferation of only a subset of strains was impaired (FIG. 13H). However, all aneuploid strains showed decreased proliferation when exposed to the protein synthesis inhibitor cycloheximide at a concentration of 0.1 μg/ml and 0.2 μg/ml, and proliferation of most was impaired at concentrations of 0.05 μg/ml (FIG. 4C). With the exception of strains disomic for chromosome I, II or IX, aneuploid strains also showed increased sensitivity to the protein synthesis inhibitors hygromycin and rapamycin (FIG. 4D, cells disomic for chromosome X were not sensitive to rapamycin, perhaps because TOR1 is located on this chromosome). The proliferation inhibitory effects of protein synthesis inhibitors on aneuploids was not a consequence of the proliferation defect of aneuploids because cdc28-4 and cdc23-1 mutants, which are severely impaired in cell division even at 23° C., did not exhibit increased sensitivity to cycloheximide, or rapamycin (FIGS. 4, C and D).

Proliferation of aneuploids was also decreased under conditions that led to the accumulation of unfolded proteins. All strains carrying an extra chromosome, with the exception of cells disomic for chromosome I, showed impaired proliferation at increased temperatures (37° C.; FIG. 4E) and were modestly sensitive to the Hsp90 inhibitor geldanamycin (except cells disomic for chromosome X, FIG. 4F).

Aneuploids did not exhibit increased sensitivity to any toxic agents. Aneuploids formed colonies as well as wild-type cells on medium containing the DNA replication inhibitor hydroxyurea (FIG. 13B), or medium containing the proline analog azetidine 2-carboxylic acid (AZC; FIG. S8E), or 6-Azauracil which interferes with UTP and GTP biosynthesis (AZA; FIG. 13I). None of the aneuploids showed altered proliferation in the presence of the autophagy inhibitors chloroquine (FIG. 13D), or hydrogen peroxide (FIG. 13G). Strains were also respiration proficient as judged by their ability to grow on the non-fermentable carbon source glycerol (FIGS. 13, C and I) and did not exhibit increased sensitivity to the FIF0 ATP synthase inhibitor oligomycin (FIG. 13I). About half of the aneuploid strains analyzed exhibited increased sensitivity to the microtubule depolymerizing drug benomyl (FIG. 13F), the basis of which warrants further investigation. These results indicate that proliferation of aneuploid strains is specifically impaired under conditions interfering with transcription, translation and protein folding.

### EXAMPLE 8

### The Phenotypes Shared by Aneuploid Yeast Strains are Due to the Presence of Additional Yeast Genes

The phenotypes shared by aneuploids might result from the mere presence of additional DNA or from the RNAs and proteins synthesized from these chromosomes. Thus, the effects of seven yeast artificial chromosomes (YACs) containing human or mouse DNA inserts ranging from approximately 350 kb to 1.6 MB in size were tested (Table 3). Although the possibility that some transcription and translation occurs from the mammalian DNA in yeast cannot be excluded, the YACs do not produce yeast proteins and it is highly likely that the amount of transcription and translation from the YACs is less than that occurring from yeast chromosomes, which are densely packed with mostly intron-less genes.

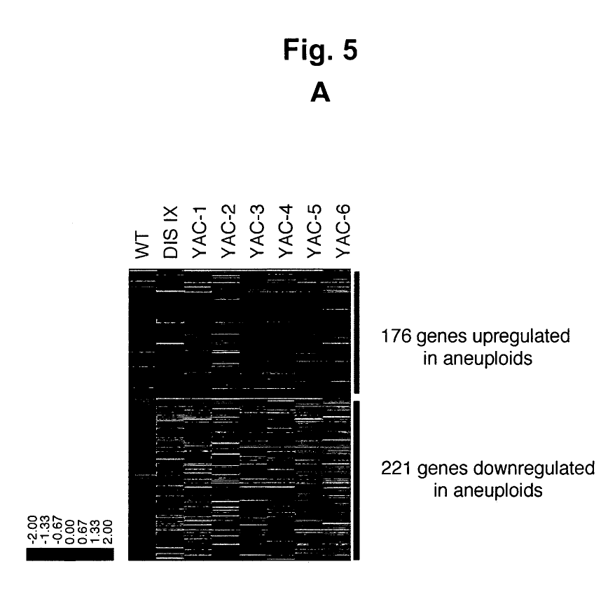

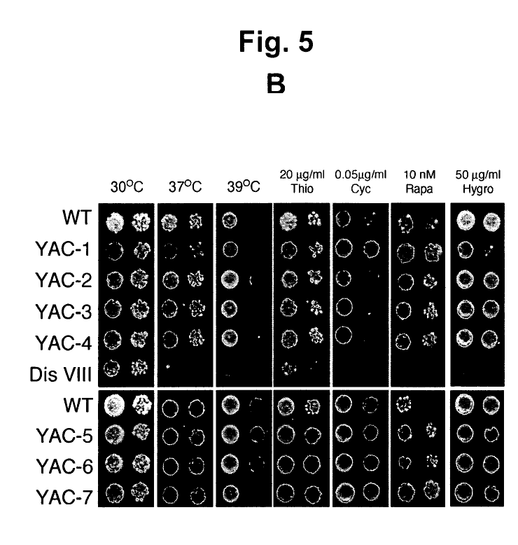

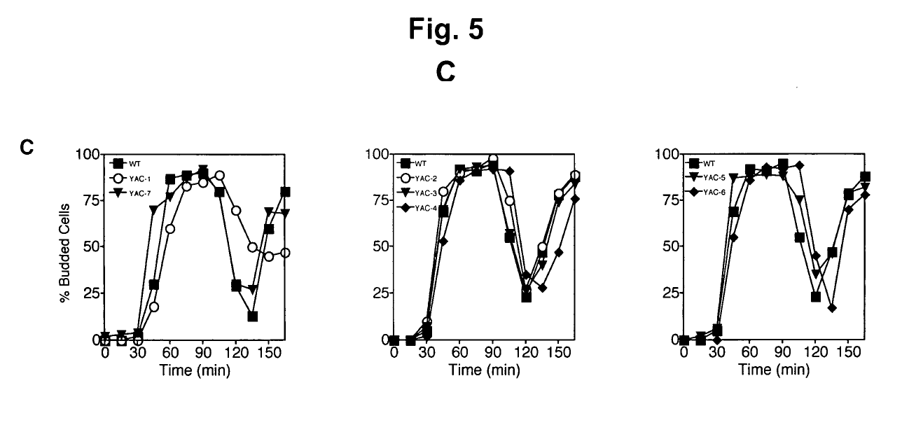

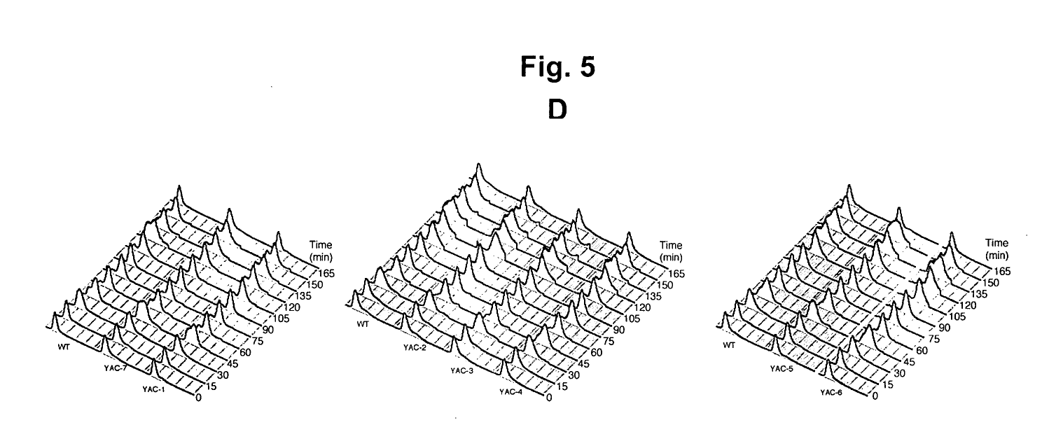

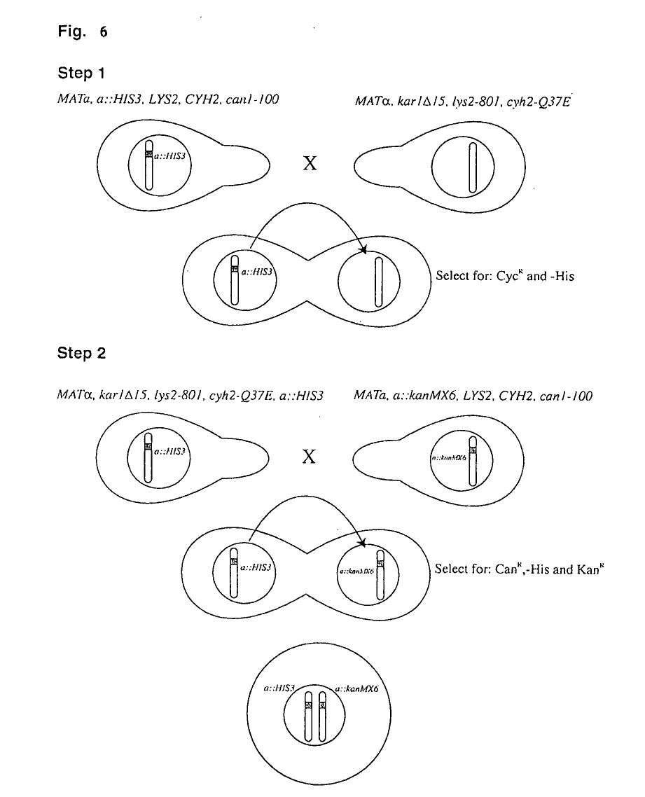

The gene expression profile shared by aneuploid strains grown under phosphate-limiting chemostat conditions was also observed in YAC-carrying strains (FIG. 5A), suggesting that the mere presence of extra DNA is mainly responsible for this gene expression pattern. The other phenotypes observed in aneuploids were not shared by the YAC-bearing strains. With the exception of a minor (5 min) delay observed in cells carrying the largest YAC (YAC-1; 1.6 MB), none of the YAC-bearing strains exhibited delays in entry into the cell cycle (FIG. 5C). Progression through other cell cycle stages was not affected either as judged by DNA content analysis (FIGS. 5, C and D). Furthermore, YAC-bearing strains did not exhibit increased sensitivity to thiolutin, cycloheximide, rapamycin, or high temperature (FIG. 5B). Curiously strain bearing the largest YAC exhibited increased sensitivity to hygromycin, the basis of which is at present unclear. These results indicate that at least two aspects of aneuploidy may contribute to the phenotypes shared by aneuploid strains: The expression signature shared by aneuploid strains appears to be elicited by the presence of extra DNA; the cell cycle delays and proliferation defects under conditions interfering with protein synthesis and folding are in large part due to the production of yeast transcripts and yeast proteins generated from extra chromosomes.

TABLE 1
Summary of cell cycle delays of aneuploid strains.

| | Strain | GI delay | G2 | | |
|---|---|---|---|---|---|
| Disome | number | (budding)^{a} | delay | Metaphase | n^{b} |
| Dis I | A12683 | 0 | 0 | 0 | 3 |
| | A6863 | 0 | 0 | 0 | 1 |
| Dis II | A12685 | 0 | 0 | 0 | 3 |
| | A6865 | 0 | 0 | 0 | 1 |
| Dis IV | A12687 | >45 | 15^{c} | 15^{c} | 3 |
| | A15232 | ≧45 | 15^{c} | 15^{c} | 1 |
| Dis V | A14479 | 0 | 15-20 | 0 | 4 |
| Dis VIII | A13628 | 10 | 0 | 0 | 3 |
| | A13629 | 10 | 0 | 0 | 1 |
| Dis IX | A13975 | 0 | 0 | 0 | 3 |
| Dis X | A12689 | 10 | 0 | 0 | 4 |
| | A6869 | 10 | 0 | 0 | 3 |
| Dis XI | A13771 | 10 | 0 | 0 | 3 |
| Dis XII | A12693 | 10-25^{d} | 10 | 0 | 4 |
| | A12694 | 15^{d} | 10 | 0 | 3 |
| Dis XIII | A12695 | 20 | 0 | 0 | 3 |
| | A12696 | 20 | 0 | 0 | 1 |
| Dis XIV | A13979 | 20 | 15 | 0 | 3 |
| Dis XV | A12697 | 5 | 0 | 0 | 3 |
| Dis XVI | A12700 | 35 | 0 | 0 | 3 |
| Dis XI + XVI | A12699 | 40 | 10 | 0 | 4 |
| Dis XI + XV | A12691 | 10 | 15 | 0 | 2 |
| Dis VIII + XIV | A15615 | 30 | 0 | 10 | 3 |
| Dis I + VI + XIII | A15619 | 30 | 0 | 0 | 2 |
| Dis VIII + XV^{e} | A15579 | 20 | 0 | 0 | 1 |
| Dis V + IX^{e} | A16308 | 15 | 15 | 0 | 1 |
| Dis V + VII^{e} | A16309 | 25 | 0 | 0 | 1 |

^{a}FACS analysis revealed similar delays.
^{b}Number of experiments.
^{c}These delays were difficult to quantify due to severity of G1 delay.
^{d}Delays were variable between different strains for disome XII.
^{e}These strains contain a small YAC (155 kb) that contains the left arm of chromosome III.

TABLE 2
Strains utilized in this study.

| Strain Number | |
|---|---|
| | Genotype |
| A702 | MATa/MATa, ade2-1, leu2-3, ura3, trp1-1, his3-11,15, |
| | can1-100, GAL, psi+ |
| A2587 | MATa, ade2-1, leu2-3, ura3, trp1-1, his3-11,15, can1-100, |
| | GAL, psi+ |
| | Relevant genotype |
| A755 | MATa, cdc23-1 |
| A2594 | MATa, cdc28-4 |
| A2596 | MATa, cdc15-2 |
| A5644* | MATc, lys2-801, cyh2, kar1 15 |
| A6844 | MATa, ade1::HIS3 |
| A6845 | MATa, ade1::KanMX6 |
| A6846 | MATa, lys2::HIS3 |
| A6847 | MATa, lys2::KanMX6 |
| A6850 | MATa, trp1::HIS3 |
| A6851 | MATa, trp1::KanMX6 |
| A6854 | MATa, ura2::HIS3 |
| A6855 | MATa, ura2::KanMX6 |
| A13576 | MATa, can1::HIS3 |
| A13624 | MATa, intergenic region (119778-119573) between YHR006W and |
| | 7C::HIS3 |
| A13625 | MATa, intergenic region (119778-119573) between |
| | YHR006W and YHR007C::KanMX6 |
| A13768 | MATa, intergenic region (430900-431000) between |
| | YKL006C-A and YKL006W::HIS3 |
| A13769 | MATa, intergenic region (430900-431000) between |
| | YKL006C-A and YKL006W::KanMX6 |
| A13972 | MATa, intergenic region (341900-34200) between YIL009W |
| | and YIL008W::HIS3 |
| A13973 | MATa, intergenic region (341900-34200) between YIL009W |
| | and YIL008W::KanMX6 |
| A13976 | MATa, intergenic region (622880-622980) between |
| | YNL005C and YNL004W::HIS3 |
| A13977 | MATa, intergenic region(622880-622980) between |
| | YNL005C and YNL004W::KanMX6 |
| A14477 | MATa, intergenic region (187520-187620) between |
| | YER015W and YER016W::KanMX6 |
| A15235 | MATa, cln2:: CLN2-3HA |
| A15548 | MATa, ade1::HIS3, lys2::KanMX6, pdr5::TRP1 |
| A15616 | MATa, met10::HIS3 |
| A15617 | MATa, met10::KanMX6 |
| A16850 | MATa, ade1::HIS3, lys2::KanMX6/YAC-1 |
| A16851 | MATa, ade1::HIS3, lys2::KanMX6/YAC-7 |
| A16854 | MATa, ade1::HIS3, lys2::KanMX6/YAC-1c |
| A17392 | MATa, ade1::HIS3, lys2::KanMX6/YAC-2 |
| A17393 | MATa, ade1::HIS3, lys2::KanMX6/YAC-3 |
| A17394 | MATa, ade1::HIS3, lys2::KanMX6/YAC-4 |
| A17396 | MATa, ade1::HIS3, lys2::KanMX6/YAC-5 |
| A17397 | MATa, ade1::HIS3, lys2::KanMX6/YAC-6 |
| A17404 | MATc, pdr5:: TRP1 |
| A17413 | MATa, leu2-3::LEU2 |
| A17414 | MATa, trp1-1:: TRP1 |
| A17415 | MATa, ura3:: URA3 |
| A17804 | MATa, cdc23-1, pdr5:: TRP1 |
| A17805 | MATa, cdc28-4, pdr5:: TRP1 |
| A17806 | MATa, cdc15-2, pdr5:: TRP1 |
| A18344 | MATa/MATa, ade1::HIS3, lys2:: URA 3, intergenic region |
| | (187520-187620) between YER015W and |
| | YER016W:: KanMX6 |

*not W303. Background unknown (likely S288c).

Aneuploid Strains

| Strain | Disomic for | |
|---|---|---|
| Number | Chromosome | |
| A6863 | I | MATa, ade1::HIS3, ade1::KanMX6 |
| A6865 | II | MATa, lys2::HIS3, lys2::KanMX6 |
| A6869 | X | MATa, ura2::HIS3, ura2::KanMX6 |
| A12683 | I | MATa, ade1::HIS3, ade1::KanMX6 |
| A12685 | II | MATa, lys2::HIS3, lys2::KanMX6 |
| A12687 | IV | MATa, trp1::HIS3, trp1::KanMX6 |
| A12689 | X | MATa, ura2::HIS3, ura2::KanMX6 |
| A12691 | XI and XV | MATa, met14::HIS3, met14::KanMX6 |
| A12693 | XII | MATa, ade16::HIS3, ade16::KanMX6 |
| A12694 | XII | MATa, ade16::HIS3, ade16::KanMX6 |
| A12695 | XIII | MATa, ura5::HIS3, ura5::KanMX6 |
| A12696 | XIII | MATa, ura5::HIS3, ura5::KanMX6 |
| A12697 | XV | MATa, leu9::HIS3, leu9::KanMX6 |
| A12699 | XI and XVI | MATa, met12::HIS3, met12::KanMX6 |
| A12700 | XVI | MATa, met12::HIS3, met12::KanMX6 |
| A13628 | VIII | MATa, intergenic region (119778-119573) between |
| | | YHR006W and YHR007C::HIS3, intergenic region |
| | | (119778-119573) |
| | | between YHR006W and YHR007C::KanMX6 |
| A13629 | VIII | MATa, intergenic region (119778-119573) between |
| | | YHR006W and YHR007C::HIS3, intergenic region |
| | | (119778-119573) |
| | | between YHR006W and YHR007C::KanMX6 |
| A13771 | XI | MATa, intergenic region (430900-431000) between |
| | | YKL006C-A and YKL006W::HIS3, intergenic region |
| | | (430900-431000) between YKL006C-A and |
| | | YKL006W:: KanMX6 |
| A13975 | IX | MATa, intergenic region (341900-34200) between |
| | | YIL009W |
| | | and YIL008W::HIS3, intergenic region (341900-34200) |
| | | between YIL009W and YIL008W::KanMX6 |
| A13979 | XIV | MATa, intergenic region (622880-622980) between |
| | | YNL005C and YNL004W::HIS3, intergenic region |
| | | (622880-622980) |
| | | between YNL005C and YNL004W::KanMX6 |
| A14479 | V | MATa, can1::HIS3, intergenic region (187520-187620) |
| | | between YER015W and YER016W:: KanMX6 |
| A15232 | IV | MATa, trp1::HIS3, trp1::KanMX6 |
| A15236 | IV | MATa, trp1::HIS3, trp1::KanMX6, cln2:: CLN2-3HA |
| A15239 | XIII | MATa, ura5::HIS3, ura5::KanMX6, cln2:: CLN2-3HA |
| A15550 | I | MATa, ade1::HIS3, ade1::KanMX6, pdr5::TRP1 |
| A15552 | II | MATa, lys2::HIS3, lys2::KanMX6, pdr5::TRP1 |
| A15554 | IV | MATa, trp1::HIS3, trp1::KanMX6, pdr5::TRP1 |
| A15556 | V | MATa, can1::HIS3, intergenic region (187520-187620) |
| | | between YER015W and YER016W::KanMX6, pdr5:: TRP1 |
| A15558 | VIII | MATa, intergenic region (119778-119573) between |
| | | YHR006W and YHR007C::HIS3, intergenic region |
| | | (119778-119573) |
| | | between YHR006W and YHR007C::KanMX6, |
| | | pdr5:: TRP1 |
| A15560 | IX | MATa, intergenic region (341900-34200) between |
| | | YIL009W |
| | | and YIL008W::HIS3, intergenic region (341900-34200) |
| | | between YIL009W and YIL008W::KanMX6, pdr5::TRP1 |
| A15562 | X | MATa, ura2::HIS3, ura2::KanMX6, pdr5::TRP1 |
| A15564 | XI | MATa, intergenic region (430900-431000) between |
| | | YKL006C-A and YKL006W::HIS3, intergenic region |
| | | (430900-431000) between YKL006C-A and |
| | | YKL006W::KanMX6, pdr5::TRP1 |
| A15566 | XII | MATa, ade16::HIS3, ade16::KanMX6, pdr5::TRP1 |
| A15567 | XIII | MATa, ura5::HIS3, ura5::KanMX6, pdr5::TRP1 |
| A15568 | XIV | MATa, intergenic region (622880-622980) between |
| | | YNL005C and YNL004W::HIS3, intergenic region |
| | | (622880-622980) |
| | | between YNL005C and YNL004W::KanMX6, |
| | | pdr5:: TRP1 |
| A15572 | XVI | MATa, met12::HIS3, met12::KanMX6, pdr5::TRP1 |
| A15579 | VIII and XV | MATa, intergenic region (119778-119573) between |
| | | YHR006W and YHR007C::HIS3, intergenic region |
| | | (119778-119573) |
| | | between YHR006W and YHR007C:: URA3/s-YAC |
| A15615 | VIII and XIV | MATa, intergenic region (119778-119573) between |
| | | YHR006W and YHR007C::HIS3, intergenic region |
| | | (119778-119573) |
| | | between YHR006W and YHR007C::KanMX6 |
| A15619 | I, VI and XIII | MATa, met10::HIS3, met10::KanMX6 |
| A16308 | V and IX | MATa, can1::HIS3, intergenic region (187520-187620) |
| | | between YER015W and YER016W:: URA3/s-YAC |
| A16309 | V and VII | MATa, can1::HIS3, intergenic region (187520-187620) |
| | | between YER015W and YER016W:: URA3/s-YAC |
| A17416 | I | MATa, ade1::HIS3, ade1::KanMX6, trp1-1:: TRP1 |
| A17417 | II | MATa, lys2::HIS3, lys2::KanMX6, ura3:: URA3 |
| A18345 | Diploid + I | MATa/MATa, ade1::HIS3, ade1::KanMX6, ade1:: URA3 |
| A18346 | Diploid + V | MATa/MATa, can1::HIS3, intergenic region (187520-187620) |
| | | between YER015W and YER016W::KanMX6, |
| | | intergenic region (187520-187620) between YER015W and |
| | | YER016W:: URA3 |
| A18347 | Diploid + VIII | MATa/MATa, intergenic region (119778-119573) between |
| | | YHR006W and YHR007C::HIS3, intergenic region |
| | | (119778-119573) |
| | | between YHR006W and YHR007C::KanMX6, |
| | | intergenic region (119778-119573) between YHR006W and |
| | | YHR007C:: URA3 |
| A18348 | Diploid + XI | MATa/MATa, intergenic region (430900-431000) between |
| | | YKL006C-A and YKL006W::HIS3, intergenic region |
| | | (430900-431000) between YKL006C-A and |
| | | YKL006W::KanMX6, intergenic region (430900-431000) |
| | | between YKL006C-A and YKL006W:: URA3 |
| A18349 | Diploid + XIV | MATa/MATa, intergenic region (622880-622980) between |
| | | YNL005C and YNL004W::HIS3, intergenic region |
| | | (622880-622980) |
| | | between YNL005C and YNL004W::KanMX6, |
| | | intergenic region (622880-622980) between YNL005C and |
| | | YNL004W:: URA3 |
| A18350 | Diploid + XV | MATa/MATa, leu9::HIS3, leu9::KanMX6, leu9::URA3 |
| A18351 | Diploid + XVI | MATa/MATa, met12::HIS3, met12::KanMX6, |
| | | met12::URA3 |

TABLE 3
List of YACs utilized in this study.

| | Approx. | | | |
|---|---|---|---|---|
| Nomenclature | Size | | Previous | |
| in this study | (kb) | DNA | Nomenclature | Reference |
| YAC-1 | 1600 | Mouse Chr X | PA3-1 | Huang et. al. |
| YAC-1 c | 800 | Mouse Chr X | PA3-1 | Truncated |
| | | | | YAC-1 |
| YAC-2 | 850 | Human Chr Y | yOX39 | Foote et.a al. |
| YAC-3 | 670 | Human Chr Y | yOX32 | Foote et.a al. |
| YAC-4 | 620 | Human Chr Y | yOX41 | Foote et.a al. |
| YAC-5 | 580 | Human Chr Y | yOX190 | Foote et.a al. |
| YAC-6 | 450 | Human Chr Y | yOX1 | Foote et.a al. |
| YAC-7 | 340 | Human Chr VII | yWSS1572-1 | Huang et. al. |
| sYAC | 155 | Left Arm | CF352 | gift from |
| | | Chr III | | F. Spencer |

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

1. L. H. Hartwell, S. K. Dutcher, J. S. Wood, B. Garvik, Rec. Adv. Yeast Mol. Biol., 28 (1982).
2. M. J. Rosenstraus, L. A. Chasin, Genetics 90, 735 (December, 1978).
3. T. Boveri, Verhandlungen der physicalisch-medizinischen Gesselschaft zu Würzburg. Neu Folge 35, 67 (1902).
4. C. Lengauer, K. W. Kinzler, B. Vogelstein, Nature 396, 643 (Dec. 17, 1998).
5. O. Niwa, Y. Tange, A. Kurabayashi, Yeast 23, 937 (Oct. 15, 2006).
6. D. L. Lindsley et al. Genetics 71, 157 (May, 1972).
7. D. J. Segal, E. E. McCoy, J Cell Physiol 83, 85 (February, 1974).
8. J. Conde, G. R. Fink, Proc Natl Acad Sci USA 73, 3651 (October, 1976).
9. T. Nilsson-Tillgren, J. G. Litske, S. Holmberg, M. C. Kielland-Brandt, Carlsberg Res. Commun. 45, 113 (1980).
10. Information on materials and methods is available on Science Online.
11. T. R. Hughes et al. Nat Biotechnol 19, 342 (April, 2001).
12. A. P. Gasch et al. Mol Biol Cell 11, 4241 (December, 2000).
13. Regenberg et al. Genome Biol 7, R107 (2006).
14. E. Hong et al. (2007).
15. M. Costanzo et al. Cell 117, 899 (Jun. 25, 2004).
16. R. A. de Bruin, W. H. McDonald, T. I. Kalashnikova, J. Yates, 3rd, C. Wittenberg, Cell 117, 887 (Jun. 25, 2004).
17. Wittenberg, K. Sugimoto, S. I. Reed, Cell 62, 225 (Jul. 27, 1990).
18. L. Dirick, T. Bohm, K. Nasmyth, Embo J 14, 4803 (Oct. 2, 1995).
19. Stuart, C. Wittenberg, Genes Dev 9, 2780 (Nov. 15, 1995).
20. L. Lehninger, D. L. Nelson, M. M. Cox, Principles of Biochemistry (Worth, New York, N.Y., ed. Second, 1993), pp. 892.
21. U. Schubert et al. Nature 404, 770 (Apr. 13, 2000).
22. Balzi, M. Wang, S. Leterme, L. Van Dyck, A. Goffeau, J Biol Chem 269, 2206 (Jan. 21, 1994).
23. P. H. Bissinger, K. Kuchler, J Biol Chem 269, 4180 (Feb. 11, 1994).
24. J. Hanna, D. S. Leggett, D. Finley, Mol Cell Biol 23, 9251 (December, 2003).
25. W. Katz, B. Weinstein, F. Solomon, Mol Cell Biol 10, 5286 (October, 1990).
26. Gunjan, A. Verreault, Cell 115, 537 (Nov. 26, 2003).
27. J. B. Easton, P. J. Houghton, Oncogene 25, 6436 (Oct. 16, 2006).
28. L. Whitesell, S. L. Lindquist, Nat Rev Cancer 5, 761 (October, 2005).
29. R. A. Gatenby, R. J. Gillies, Nat Rev Cancer 4, 891 (November, 2004).
30. B. A. Weaver, D. W. Cleveland, Curr Opin Cell Biol 18, 658 (December, 2006).
31. M. J. Dunham et al, Proc Natl Acad Sci USA 99, 16144 (Dec. 10, 2002).
S1. M. Tyers, G. Tokiwa, B. Futcher, Embo J 12, 1955 (May, 1993).
S2. M. S. Longtine et al. Yeast 14, 953 (July, 1998).
S3. Y. Hugerat, F. Spencer, D. Zenvirth, G. Simchen, Genomics 22, 108 (Jul. 1, 1994).
S4. J. Conde, G. R. Fink, Proc Natl Acad Sci USA 73, 3651 (October, 1976).
S5. T. Nilsson-Tillgren, J. G. Litske, S. Holmberg, M. C. Kielland-Brandt, Carlsberg Res. Commun. 45, 113 (1980).
S6. N. F. Kaufer, H. M. Fried, W. F. Schwindinger, M. Jasin, J. R. Warner, Nucleic Acids Res 11, 3123 (May 25, 1983).
S7. H. Liu, J. Krizek, A. Bretscher, Genetics 132, 665 (November, 1992).
S8. W. Katz, B. Weinstein, F. Solomon, Mol Cell Biol 10, 5286 (October, 1990).
S9. B. Weinstein, F. Solomon, Mol Cell Biol 10, 5295 (October, 1990).
S10. D. Huang, D. Koshland, Genes Dev 17, 1741 (Jul. 15, 2003).
S11. S. Foote, D. Vollrath, A. Hilton, D. C. Page, Science 258, 60 (Oct. 2, 1992).
S12. A. Amon, U. Surana, I. Muroff, K. Nasmyth, Nature 355, 368 (Jan. 23, 1992).
S13. O. Cohen-Fix, J. M. Peters, M. W. Kirschner, D. Koshland, Genes Dev 10, 3081 (Dec. 15, 1996).
S14. C. B. Epstein, F. R. Cross, Genes Dev 6, 1695 (September, 1992).
S15. R. Visintin, S. Prinz, A. Amon, Science 278, 460 (Oct. 17, 1997).
S16. F. C. Holstege et al. Cell 95, 717 (Nov. 25, 1998).
S17. S. Ghaemmaghami et al. Nature 425, 737 (Oct. 16, 2003).
S18. N. N. Batada et al. PLoS Biol 4, e317 (September, 2006).

What is claimed is:

1. A method for facilitating the diagnosis cancer or a predisposition thereto in a subject, comprising detecting a presence or an absence of a mutation in
a) a gene encoding a Ubiquitin-specific protease polypeptide;
b) a gene encoding a DNA helicase polypeptide;
c) a gene encoding a Subunit of the Set3C deacetylase complex polypeptide or
d) a promoter of ORF YJL213W or a human homologue thereof
wherein the presence of said mutation indicates the presence of a cancer or a predisposition thereto in said subject.

2. The method of claim 1, wherein said mutation in the gene encoding the Ubiquitin-specific protease polypeptide results in a C-terminal truncation of the polypeptide and a decrease in peptidase activity of the polypeptide.

3. The method of claim 1, wherein said mutation in the gene encoding the Ubiquitin-specific protease polypeptide results in a stop codon.

4. The method of claim 1, wherein mutation in the gene encoding the DNA helicase polypeptide or Subunit of the Set3C deacetylase complex polypeptide results in a alteration in an activity of the polypeptide.

5. The method of claim 1, wherein said mutation in the gene encoding the DNA helicase polypeptide or Subunit of the Set3C deacetylase complex polypeptide is a point mutation.

6. The method of claim 5, wherein said point mutation in the gene encoding the DNA helicase polypeptide results in an amino acid change at position 148.

7. The method of claim 6, wherein said amino acid change is an aspartic acid to an asparagine.

8. The method of claim 5, wherein said point mutation in the gene encoding the Subunit of the Set3C deacetylase complex polypeptide results in an amino acid change at position 431.

9. The method of claim 8, wherein said amino acid change is a leucine to an arginine.

10. The method of claim 1, wherein mutation in the promoter of ORF YJL213W or a human homologue thereof results in increased expression of the polypeptide encode by ORF YJL213W or the human homologue.

11. The method of claim 1 wherein said gene a gene encoding a Ubiquitin-specific protease polypeptide is UBP6 or a human homologue thereof.

12. The method of claim 11, wherein said human homologue is USP14.

13. The method of claim 1 wherein said gene a gene encoding a DNA helicase polypeptide is RAD3 or a human homologue thereof.

14. The method of claim 13, wherein said human homologue is ERCC2.

15. The method of claim 1 wherein said gene a gene encoding a Subunit of the Set3C deacetylase complex polypeptide is SNT1 or a human homologue thereof.

16. The method of claim 15, wherein said human homologue is FRS2 or SNTA1.

17. A method according to claim 1, wherein said sample is serum, blood plasma, ascites fluid, urine, or tissue biopsy.

18. The method of claim 1, wherein said mutation is detected electrophoretically, or immunochemically.

19. The method of claim 1, wherein said mutation is determined by a method selected from the group consisting of polymerase chain reaction, single nucleotide polymorphism (SNP) arrays, and interphase fluorescent in situ hybridization (FISH) analysis.

20. A method according to claim 1, wherein said subject has not been previously diagnosed as having cancer.

21. A method according to claim 1, wherein said subject has been previously diagnosed as having cancer.

22. A method of decreasing cancer cell growth comprising contacting said cancer cell with a glucose transporter inhibitor, an RNA polymerases inhibitor, a protein synthesis inhibitor or a HSP90 inhibitor.

23. The method of claim 21, wherein said protein synthesis inhibitor is cycloheximide, hygromycin or rapamycin.

24. The method of claim 21 wherein said HSP90 inhibitor is geldanamycin.

25. The method of claim 21, wherein said cell is aneuploid.

26. A method of decreasing cancer cell growth comprising contacting said cancer cell with a compound that:
a) increases the expression or activity of a non-mutated UBP6, RAD3 or SNT1 polypeptide or human homologue thereof,
b) decrease intracellular protein degradation; or
c) decreases the expression of activity of the polypeptide encoded by ORF YJL213W or a human homologue thereof.

27. A method for screening for an inhibitor of tumor cell proliferation or viability, comprising
a) contacting an aneuploid cell with a candidate compound, and
b) measuring cell proliferation or viability, wherein a decrease in aneuploid cell proliferation or viability in the presence of said compound, as compared to the absence of the compound, indicates that the compound is an inhibitor of tumor cell proliferation or viability.

28. The method of claim 28, wherein said aneuploid cell displays wild-type physiology.

29. The method of claim 28, wherein said aneuploid cell has at least one mutation in a nucleic acid selected from the group consisting of:
a) a Ubiquitin-specific protease gene;
b) a DNA helicase gene;
c) a Subunit of the Set3C deacetylase complex gene or
d) a promoter of ORF YJL213W or a human homologue thereof

30. A method for screening for a specific inhibitor of cancer cell proliferation or viability, comprising:
a) contacting an aneuploid cell wherein said aneuploid cell has at least one mutation in a nucleic acid selected from the group consisting of a Ubiquitin-specific protease gene, a DNA helicase gene, a Subunit of the Set3C deacetylase complex gene and a promoter of ORF YJL213W or a human homologue thereof with a candidate compound,
b) contacting a diploid cell with the candidate compound;
c) measuring cell proliferation or viability, wherein a decrease in aneuploid cell proliferation or viability, as compared to the diploid cell indicates that the compound is a specific inhibitor of tumor cell proliferation or viability.
